(19) United States
(12) Patent Application Publication
(10) Pub. No.: US 2021/0077728 A1
(43) Pub. Date: Mar. 18, 2021

(54) NEEDLE-FREE INJECTOR FOR LARGE-SCALE, MULTI-DOSE APPLICATIONS

(71) Applicant: PORTAL INSTRUMENTS, INC., Cambridge, MA (US)

(72) Inventors: Patrick Armand Anquetil, Cambridge, MA (US); Andrew Owen Coats, Somerville, MA (US); Robert Joseph Dyer, Cambridge, MA (US); David Siet, Cambridge, MA (US)

(21) Appl. No.: 17/025,768

(22) Filed: Sep. 18, 2020

Related U.S. Application Data
(60) Provisional application No. 62/902,169, filed on Sep. 18, 2019.

(51) Int. Cl. A61M 5/30; A61M 5/19; A61M 5/46; A61D 1/02; A61D 7/00

(52) U.S. Cl. CPC A61M 5/30; A61M 5/19; A61M 5/46; A61M 2250/00; A61D 7/00; A61M 2205/6054; A61M 2205/502; A61D 1/025

(57) ABSTRACT

A needle-free injector includes a housing, a cartridge positioned within the housing, and a plunger slidably coupled to and disposed within the chamber, a motor operatively coupled to the plunger, the motor operable to actuate the plunger in the chamber, and a controller operatively coupled to the motor. Methods of delivering an injectate using the needle-free injectors are provided. Methods of facilitating needle-free injection of a fluid using the needle-free injectors are also provided.

### CROSS-REFERENCE TO RELATED APPLICATIONS

This application claims priority under 35 U.S.C. § 119(e) to U.S. Provisional Patent Application Ser. No. 62/902,169 titled “Needle-Free Injection” filed Sep. 18, 2019, the entire disclosure of which is hereby incorporated herein by reference in its entirety for all purposes.

### FIELD

This disclosure generally relates to needle-free injection, and more particularly to injectors for large scale, multi-dose, multi-target applications such as drug delivery for cows, pigs, sheep and other livestock.

### BACKGROUND

Humans and livestock often receive drugs, supplements and the like in formulations intended for injection. However, the use of needles for injections presents significant drawbacks ranging from difficulties in handling to safety hazards associated with the needles themselves. Needle-free transdermal injection devices have been developed as an alternative to needle-based injectors. However, there remains a need for improved needle-free injectors adapted for use in human and agricultural contexts.

### SUMMARY

A handheld needle-free injector is disclosed for large scale applications such as emergency or rapid human immunization and livestock immunization. Needle-free injectors disclosed herein provide for precise control over numerous aspects of the injection process, resulting in reduced pain experienced by the subjects being treated, improved operator safety, and efficient use of injectates. Other features and advantages of the invention are apparent from the following description, and from the claims.

In accordance with an aspect, there is provided a needle-free injector. The needle free injector may include a housing, at least one chamber within the housing constructed and arranged to be fluidly coupled to at least one source of an injectate, and at least one nozzle fluidly coupled to the chamber. The needle-free injector may further include a plunger slidably coupled to and disposed within the chamber, and a motor operatively coupled to the plunger. The plunger may be positioned to discharge a bolus of the at least one injectate through an exit port when slid within the chamber. The motor may be operable to actuate the plunger in the chamber. The needle-free injector may additionally include a controller operatively coupled to the motor. The controller may be operable to operate the plunger according to a first delivery profile configured to inject a first bolus of a first injectate to a first predetermined depth in a subject. The controller may be further operable to operate the plunger according to a second delivery profile configured to inject a second bolus of a second injectate to a second predetermined depth in the subject. The first predetermined depth and the second predetermined depth may be such that the first bolus and the second bolus do not mix at the injection site upon injection into the subject.

In some embodiments, the predetermined depth for each of the boluses of the first injectate and second injectate delivered may be determined by at least one of a velocity of each injectate, an identity of a subject, a species of the subject, a delivery location on the subject, a prescribed regimen for the subject, and a composition of the volume of each injectate.

In further embodiments, the needle-free injector may include a plurality of chambers constructed and arranged to be fluidly coupled to a respective plurality of sources of injectates. The plurality of chambers may be configured to accept a plurality of plungers disposed within the respective plurality of chambers. For example, the controller may be operable to operate the plurality of plungers according to a plurality of delivery profiles.

In some embodiments, the at least one nozzle may include at least one of an adjustable exit diameter and an orientation that is adjustable relative to an axis of flow through the plurality of chambers. In such implementations, each of the plurality of delivery profiles may be configured to adjust at least one of the nozzle exit diameter and nozzle orientation responsive to at least one of desired injectate velocity, an identity of a subject, a species of the subject, a delivery location on the subject, a prescribed regimen for the subject, a subset of a plurality of the subjects, and a composition of each of the plurality of injectates.

In further embodiments, the needle-free injector may include a sensor configured to acquire a signal from a detectable marker. For needle-free injectors including a sensor, the controller may be further operable to operate the plunger according to the first delivery profile responsive at least to receiving a signal from the detectable marker.

In further embodiments, the needle-free injector may include a marking system configured to apply an identifier on the subject. For needle-free injectors including a marking system, the controller may be further operable to cause the marking system to apply the identifier to the subject responsive to delivering the bolus of the injectate to the subject.

In accordance with an aspect, there is provided a needle-free injector. The needle-free injector may include a housing, a plurality of chambers within the housing constructed and arranged to be fluidly coupled to a respective plurality of sources of an injectate, and a plurality of nozzles fluidly coupled to the plurality of chambers. Each of the plurality of nozzles may be associated with a respective one of the plurality of chambers and spaced to prevent mixing of injectates. Each of the plurality of nozzles may include an adjustable exit diameter and an orientation that is adjustable relative to an axis of flow through the plurality of chambers. The needle-free injector may include a plurality of plungers disposed within a respective plurality of chambers and a motor operatively coupled to the plurality of plungers. Each of the plurality of plungers may be adapted to slide within a respective chamber and discharge a bolus of one of the plurality of injectates from each of the plurality of chambers. The motor may be operable to actuate the plurality of plungers in the plurality of chambers. The needle-free injector may additionally include a controller operatively coupled to the motor. The controller may be operable to selectively operate the plurality of plungers according to a plurality of delivery profiles, where each of the plurality of delivery profiles may be associated with a respective one of the plurality of chambers and configured to adjust at least one of the nozzle exit diameter and nozzle orientation responsive to at least an identity of a subject, a delivery location on the subject, a velocity of the injectate associated with each of the plurality of chambers, and a composition of each of the plurality of injectates.

In further embodiments, the needle-free injector may include a sensor configured to acquire a signal from a detectable marker. For needle-free injectors including a sensor, the controller may further operable to operate the plunger according to the first delivery profile responsive at least to receiving a signal from the detectable marker.

In further embodiments, the needle-free injector may include a marking system configured to apply an identifier on the subject. For needle-free injectors including a marking system, the controller may be further operable to cause the marking system to apply the identifier to the subject responsive to delivering the bolus of the injectate to the subject.

In some embodiments, the exit diameter of at least one of the plurality of nozzles may be different than the remainder of the plurality of nozzles. In some embodiments, each of the plurality of nozzles may have the same exit diameter. In certain embodiments, each of the plurality of nozzles may have a different exit diameter.

In some embodiments, the controller may be further operable to operate the plurality of plungers according to a plurality of delivery profiles configured to inject the bolus of the injectate from the respective plurality of chambers to a predetermined depth. In such implementations, the predetermined depth for each of the injectates may be chosen such that injectates do not mix in a tissue of the subject upon injection into the subject. For example, the predetermined depth for each of the boluses of injectates injected may be determined by at least at least one of a desired velocity of each injectate, an identity of a subject, a species of the subject, a delivery location on the subject, a prescribed regimen for the subject, a subset of a plurality of the subjects, and a composition of the bolus of each of the plurality of injectates.

In accordance with an aspect, there is provided a needle-free injector. The needle-free injector may include a housing, a chamber within the housing constructed and arranged to be fluidly coupled to at least one source of an injectate, at least one nozzle fluidly coupled to the chamber, and a plunger slidably coupled to and disposed within the chamber. The plunger may be positioned to discharge a bolus of injectate from the at least one source of the injectate through an exit port when slid within the chamber. The needle-free injector may further include a motor operatively coupled to the plunger, a sensor configured to acquire a signal from a detectable marker, and a marking system configured to apply an identifier on a subject. The motor may be operable to actuate the plunger in the chamber. The needle-free injector may additionally include a controller operatively coupled to the motor. The controller may be operable to selectively operate the plunger according to a delivery profile configured to deliver the bolus of the injectate. Operation of the delivery profile may be responsive at least to receiving a signal from the detectable marker, with the controller further operable to cause the marking system to apply the identifier to the subject responsive to injecting the bolus of the injectate to the subject.

In some embodiments, the chamber may be configured to be fluidly coupled to a plurality of sources of injectate. In further embodiments, the needle-free injector may include a plurality of chambers.

In some embodiments, the at least one nozzle may include at least one of an adjustable exit diameter and an orientation that is adjustable relative to an axis of flow through the chamber. In such implementations, the controller may be further operable to adjust at least one of the nozzle exit diameter and nozzle orientation responsive to at least one of an identity of a subject, a species of the subject, a delivery location on the subject, a prescribed regimen for the subject, a subset of a plurality of the subjects, and a composition of the bolus of the injectate. In particular embodiments, the adjustment to at least one of the nozzle exit diameter and nozzle orientation may be responsive to at least velocity of the injectate, an identity of a subject, a delivery location on the subject, and a composition of the volume of the bolus of the injectate.

In further embodiments, the controller may be operable to operate the plunger to deliver a plurality of boluses of injectates from the respective plurality of chambers to a predetermined depth. For example, the predetermined depth for each of the injectates may be such that each bolus delivered does not mix at the injection site upon injection into the subject. In such cases, the predetermined depth for each of the boluses of the injectates may be determined by at least a velocity of the injectate, an identity of a subject, a delivery location on the subject, and a composition of the bolus of the injectate.

In some embodiments, the detectable marker includes an electromagnetic emitter, for example a radio frequency identification (RFID) tag. In some embodiments, the detectable marker comprises a fiducial marker, for example a barcode.

In further embodiments of any needle-free injector disclosed herein, the source of the injectate may include a reservoir fixedly connected to the housing. In further embodiments of any needle-free injector disclosed herein, a source of the injectate may include a reservoir fluidly coupled to the housing by a flexible conduit.

In further embodiments of any needle-free injector disclosed herein, the needle-free injector may include at least one light source constructed and arranged to illuminate a mark, such as a crosshair, onto an area of the subject.

In further embodiments of any needle-free injector disclosed herein, the needle-free injector may include a graphical display integrated into the housing operatively coupled to the controller. The graphical display may be configured to display to a user information pertaining to at least one of the status of the needle-free injector and an injection process. For example, the graphical display may be configured to display a status of the needle-free injector that includes at least one of a remaining charge on a battery and service messages for the needle-free injector. In some embodiments, the graphical display may be configured to display at least one of a number of subjects who have received injections, information extracted from a detectable marker, a volume of fluid remaining in the at least one source of injectate and a number of subjects remaining to receive a volume of fluid.

In further embodiments of any needle-free injector disclosed herein, the controller of the needle-free injector may be operable to initiate a cleaning cycle responsive to a final delivery of injectate, the cleaning cycle comprising discharging a volume of a fluid through the at least one nozzle.

In further embodiments of any needle-free injector disclosed herein, the controller of the needle-free injector may be operable to collect and store data pertaining to at least information extracted from the detectable marker, number of subjects who have received injections, date and time of delivered injections, composition of the injectate, and an aggregate volume of injectate injected.

In accordance with an aspect, there is provided a method of delivering a fluid using a needle-free injector. The method may include providing the needle-free injector as described herein and responsive to initiating an injection with the needle-free injector, causing the needle-free injector to inject a bolus of an injectate into a subject.

In further embodiments, the method may include, prior to initiating an injection, identifying a subject in need thereof by extracting information from a detectable marker coupled to the subject.

In further embodiments, the method may include measuring an aggregate volume of injectate delivered.

In further embodiments, the method may include measuring an aggregate count of the number of injectates delivered to a plurality of subjects. For example, following measuring an aggregate count of the number of injectates delivered to a plurality of subjects, the method may include subtracting the aggregate count from an expected total count to determine a remaining population of subjects requiring injection.

In further embodiments, the method may include applying an identifier to the subject responsive to injecting the subject.

In accordance with an aspect, there is provided a method of facilitating needle-free injection of a fluid. The method may comprise providing a needle-free injector as disclosed herein.

In further embodiments, the method may include providing instructions to a user for connecting at least one source of the fluid to the needle-free injector. In further embodiments, the method may include providing instructions to a user for operating the needle-free injector.

### BRIEF DESCRIPTION OF DRAWINGS

FIG. 1 is a schematic diagram of a controllable, needle-free transdermal injection device;

FIG. 2 is a cut-away diagram of a ball screw actuator;

FIG. 3 is a block diagram of the controllable, needle-free transdermal injection device of FIG. 1;

FIG. 4 is a detailed block diagram of the controllable, needle-free transdermal injection device of FIG. 1;

FIG. 5 is a detailed block diagram of the power supply of the controllable, needle-free transdermal injection device of FIG. 1;

FIG. 6 is a target displacement profile;

FIG. 7 is a rotary motor speed profile associated with the target displacement profile of FIG. 6;

FIG. 8 is an injectate jet velocity profile associated with the target displacement profile of FIG. 6;

FIGS. 9-19 show various views of embodiments of needle-free injection devices;

FIG. 20 illustrates a schematic of a needle-free transdermal injection device having dual actuators, according to one embodiment;

FIG. 21 illustrates a pair of needle-free transdermal injection devices delivering an injectate to a porcine subject, according to one embodiment; and

FIG. 22 illustrates a schematic view of a controllable, needle-free transdermal injection device, according to one embodiment.

### DETAILED DESCRIPTION

In the following document, references to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context. Thus, the term “or” should generally be understood to mean “and/or” and so forth.

Recitation of ranges of values herein are not intended to be limiting, referring instead individually to any and all values falling within the range, unless otherwise indicated, and each separate value within such a range is incorporated into the specification as if it were individually recited herein. The words “about,” “approximately” or the like, when accompanying a numerical value or physical property, are to be construed as indicating a deviation as would be appreciated by one of ordinary skill in the art to operate satisfactorily for an intended purpose. Similarly, words of approximation such as “approximately” or “substantially” when used in reference to physical characteristics, should be understood to contemplate a range of deviations that would be appreciated by one of ordinary skill in the art to operate satisfactorily for a corresponding use, function, purpose or the like. Ranges of values and/or numeric values are provided herein as examples only, and do not constitute a limitation on the scope of the described embodiments unless explicitly stated otherwise. The use of any and all examples, or exemplary language (“e.g.,” “such as,” or the like) provided herein, is intended merely to better illuminate the embodiments and does not pose a limitation on the scope of the embodiments. No language in the specification should be construed as indicating any unclaimed element as essential to the practice of the embodiments. In the following description, it is understood that terms such as “first,” “second,” “top,” “bottom,” “up,” “down,” and the like, are words of convenience and are not to be construed as limiting terms.

### Needle-Free Transdermal Injection Device

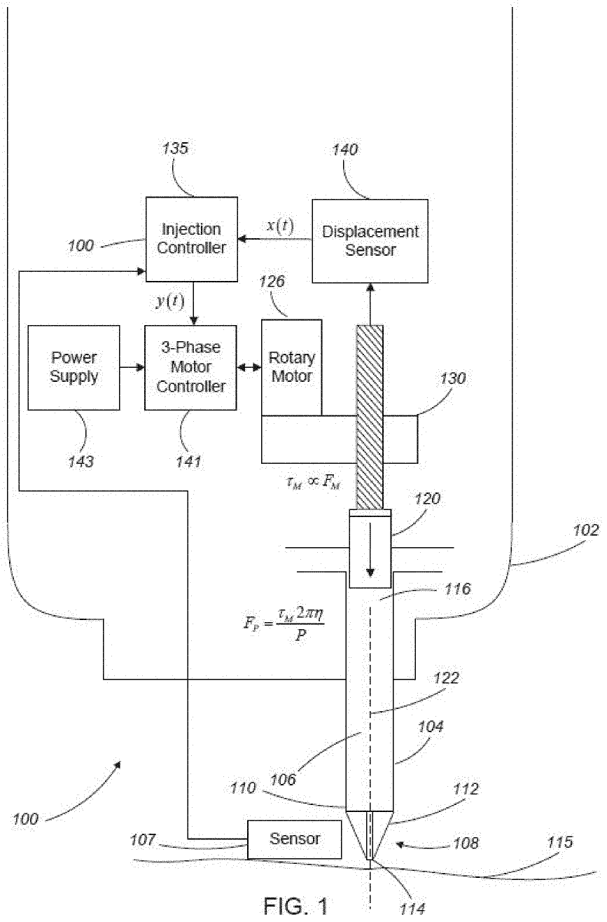

Referring to FIG. 1, a controllable, needle-free transdermal injection device 100 for transferring an injectate (e.g., a drug or a vaccine in any one of a number of states such as a liquid state or a powder state) through the skin of a patient includes a needle-free transdermal injector head 104 extending from a housing 102. The injector head 104 includes a chamber 106 for holding the injectate and a nozzle 108 disposed at a distal end 110 of the injector head 104. The nozzle 108 includes a head 112 and an opening 114 from which a jet of the injectate is discharged from the chamber 106. In operation, the opening 114 is placed near or against the skin 115 when the injectate is discharged. The dimensions of the nozzle 108 may be adapted to control a shape and pressure profile of a stream of injectate exiting the nozzle 108. For example, the inner diameter of the opening 114 may be in a range of 50 μm to 300 μm, and may employ a taper along the longitudinal axis 122 toward the opening to shape an exiting stream of injectate. It will also be appreciated that the geometry of the chamber 106 relative to the opening 114 may affect how linear motion of a plunger or the like within the chamber 106 translates into an exit velocity or pressure by an injectate through the opening 114. An outer diameter of the head 112 of the nozzle 108 may narrow to the opening 114, or may remain uniform or may expand to provide a suitable resting surface for the head 112 of the nozzle 108. The nozzle 108 may have a length along the longitudinal axis 122 of about 500 μm to about 5 mm. Similarly, the chamber 106 may have any suitable length along the longitudinal axis for containing an injectate, and for displacing the injectate through the opening 114 in one or more needle-free injections.

The chamber 106 may have a proximal end 116 and a distal end 110. An actuator (i.e., a piston or plunger 120) may be slidably disposed within the chamber 106. Movement of the plunger 120 along a longitudinal axis 122 in either direction can affect the pressure within chamber 106. In some embodiments, the chamber 106 is integral to the device 100. In other embodiments, the chamber 106 is separately attachable to device 100.

In some examples, the injection device 100 includes a sensor 107 (e.g., a mechanical sensor or a capacitive sensor) for detecting a contact between the apparatus and the skin of a patient. In some examples, the sensor 107 is configured to detect an angle of the cartridge relative to the skin of the patient. In some examples, the sensor 107 is configured to detect a position of the injection opening relative to the patient's skin 115 or body. In some examples, the sensor 107 communicates with the injection controller 100 to prevent injection from occurring when the apparatus is not in contact with the patient's skin 115 or when an angle or position of the apparatus relative to the patient is incorrect.

### Rotary Motor

In general, needle-free injectors disclosed herein are constructed and arranged to use a motor to drive the plunger within a chamber to deliver an injectate. In contrast, typical injectors, such as the powered injector described in U.S. Pat. No. 10,434,257, operate using compressed air to deliver the injectate from the injector. Compressed air driven devices require attachment to an air compressor or other suitable source of a pressurized gas, which may provide for excessive noise levels when in operation. Excessive noise may be an issue in animal husbandry operations, such as by startling or stressing livestock, and may cause difficulties in delivering the injectates to the subjects in need thereof. Conversely, human subjects may be stressed by excessive device noise. Electric motors, such as those used in needle-free injectors disclosed herein, provide for reduced noise operation due to their constriction, which may have benefits for injection processes in crowded animal husbandry and farm operations.

The injection device 100 may include an electromagnetic rotary motor 126 that applies a force to the plunger 120 via a linkage 130 to inject the injectate in the chamber 106 through the skin 115. The linkage may include a ball screw actuator 130, and the linkage may also or instead include any other suitable mechanical coupling for transferring a rotary force of the rotary motor 126 into a linear force suitable for displacing injectate from the chamber 106. For example, the linkage may include one or more of lead screws, linear motion bearings, and worm drives, or another other suitable mechanical components or combination of mechanical components. As noted above, linear motion may usefully be inferred from rotation of a lead screw or the like, and the injection device 100 may be instrumented to monitor rotation in order to provide feedback on a position of the plunger 120 to a controller during an injection.

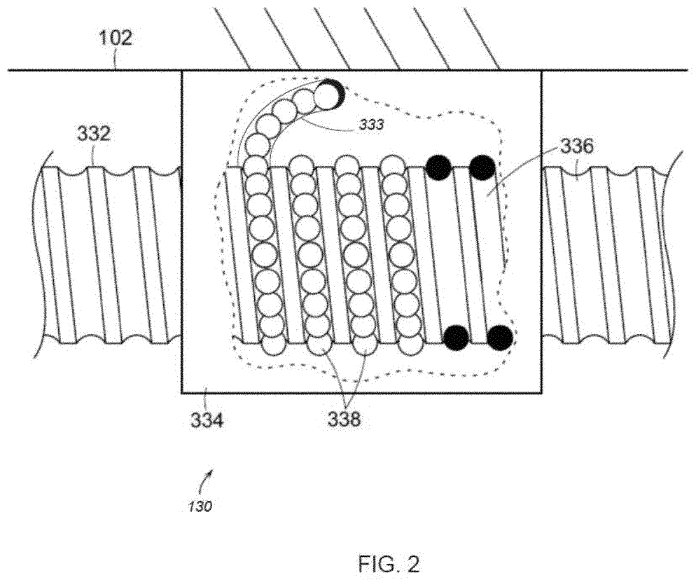

Referring to FIG. 2, one example of a ball screw actuator 130 includes a screw 332 and a nut 334 (which is coupled to the housing 102 in FIG. 1), each with matching helical grooves 336. The ball screw actuator 130 may include a recirculating ball screw with a number of miniature balls 338 or similar bearings or the like that recirculate through the grooves 336 and provide rolling contact between the nut 334 and the screw 332. The nut 334 may include a return system 333 and a deflector (not shown) which, when the screw 332 or nut 334 rotates, deflects the miniature balls 338 into the return system. The balls 338 travel through the return system to the opposite end of the nut 334 in a continuous path. The balls 338 then exit from the ball return system into the grooves 336. In this way, the balls 338 continuously recirculate in a closed circuit as the screw 332 moves relative to the nut 334.

In some examples, the rotary motor 126 is of a type selected from a variety of rotational electrical motors (e.g., a brushless DC motor). The rotary motor 126 is configured to move the screw 332 of the ball screw actuator 130 back and forth along the longitudinal axis 122 by applying a torque (i.e., τ_{M}) to either the screw 332 or the nut 334 of the ball screw actuator. The torque causes rotation of either the screw 332 or the nut 334, which in turn causes an input force F_{M}(t), which is proportional to the torque applied by the motor, to be applied to the screw 332.

The torque τ_{M} applied to the screw 332 causes application of a force F_{P} to the plunger 120 which in turn causes movement of the plunger 120 along the longitudinal axis 122. The force F_{P} is determined according to the following equation representing an idealized relationship between torque and force for a ball screw actuator:

$${F}_{P} = \frac{{τ}_{M} 2 π η}{P}$$

where F_{P} is a force applied to the plunger 120 by the screw 332, τ_{M} is a torque applied to the screw 332, η is an efficiency of the ball screw actuator 130, and P is a lead of the screw 332.

### Control Loop

Referring again to FIG. 1, the transdermal injection device 100 may include a displacement sensor 140, an injection controller 135, and a three-phase motor controller 141. In general, the displacement sensor 140 measures a displacement x(t) of the screw 332 of the ball screw actuator 130 and/or the plunger 120. The displacement sensor 140 may, for example, measure an incremental displacement of the screw 332 by storing an initial displacement value (i.e., x(0)) and monitoring a deviation from the starting value over time. In other examples, the displacement sensor 140 measures an absolute displacement of the screw 332 relative to a position of the displacement sensor 140 or some other fixed reference point. In another aspect, the displacement sensor 140 may be coupled to a nut or other component of a ball screw that controls linear movement. In this configuration, the displacement sensor 140 can measure rotation of the screw drive, and rotational motion may be computationally converted into linear displacement for purposes of controlling operation of the device 100.

The displacement x(t) measured by (or calculated using data from) the displacement sensor 140 may be provided as input to the injection controller 135. As is described in greater detail below, the injection controller 135 processes the displacement x(t) to determine a motor control signal y(t). The motor control signal y(t) is provided to the three-phase motor controller 141 which, in conjunction with a power supply 143, drives the rotary motor 126 according to the motor control signal y(t). The motor 126 causes the torque τ_{M} (t) to be applied to the screw 332. The motor torque, τ_{M}(t) causes movement of the screw 332 (or any other suitable linear actuator) in a direction along the longitudinal axis 122.

### System Diagram

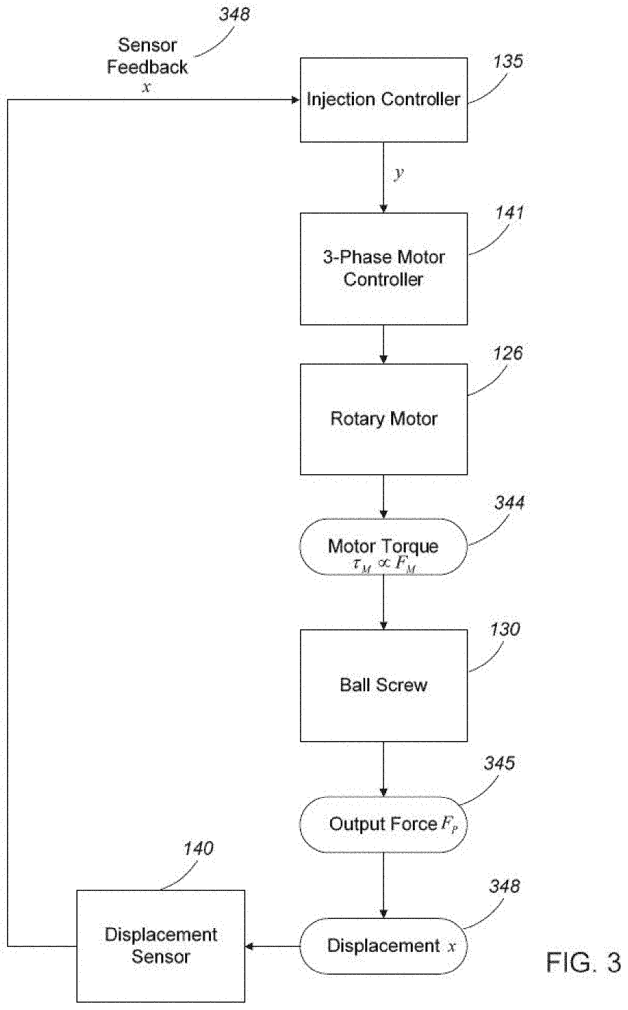

Referring to FIG. 3, a schematic diagram of the system of FIG. 1 shows the rotary motor torque τ_{M} being applied to the ball screw 130 in step 344. Application of the rotary motor torque, at a given time 11 by the rotary motor causes application of a force, F_{M}(t₁) to the screw 332 of the ball screw 130 as shown in step 345, which in turn causes a displacement of the screw 332 in step 348.

The displacement of the screw 332 of the ball screw 130 is measured by the displacement sensor 140 and is fed back to the injection controller 135. As is described in greater detail below, the injection controller 135 processes the measured displacement to provide sensor feedback 348 to determine a motor control signal y(t₁) which is supplied to the three-phase motor controller 141. The three-phase motor controller 141 drives the rotary motor 326 according to the motor control signal y(t₁), causing the motor 126 to apply a torque τ_{M} (t₂) to the screw 332 of the ball screw 130 at a time t₂. As is noted above, the torque τ_{M} applied to the screw 332 causes application of a force F_{P} to the plunger 120 with F_{P} being determined as:

$${F}_{P} = \frac{{τ}_{M} 2 π η}{P}$$

where F_{P} is a force applied to the plunger 120 by the screw 332, τ_{M} is a torque applied to the screw 332, η is an efficiency of the ball screw actuator 130, and P is a lead of the screw 332.

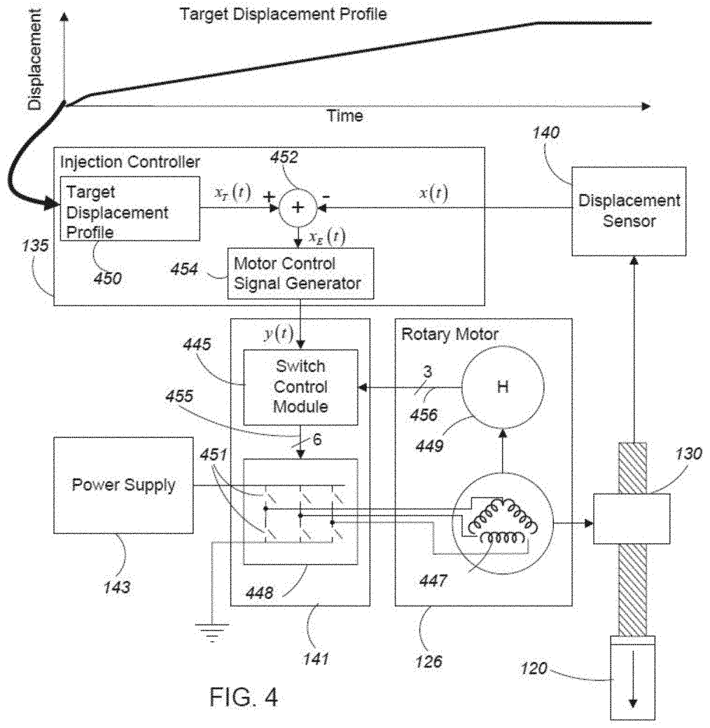

Referring to FIG. 4, in some examples the injection controller 135 includes a target displacement profile 450, a summing block 452, and a motor control signal generator 454. Very generally, the injection controller 135 receives a displacement value x(t) at time t from the displacement sensor 140. The time t is provided to the target displacement profile 450, which determines a target displacement value x_{T}(t) for the time 1.

In some examples, the target displacement profile 450 includes a mapping between target displacement values and times associated with an injection cycle (i.e., a range of time over which the plunger 120 of the device moves). For example, in the target displacement profile 450 shown in FIG. 4 the displacement starts at zero at the beginning of an injection cycle (i.e., at time t₀) and changes (e.g., increases) over time as the injection cycle proceeds, with each instant in time of the injection cycle being associated with a corresponding displacement value. As is described in greater detail below, in some examples the rate of change of the displacement values varies over time, with different time intervals of the injection cycle being associated with different rates of change of displacement values. Control of the plunger displacement, e.g., according to the target displacement profile 450, can be used to perform complex injections. For example, in one aspect, the plunger 120 is displaced relatively quickly during an initial piercing phase to penetrate the skin barrier, and in other time intervals the plunger 120 is displaced relatively slowly to deliver the injectate through an opening formed during the initial, piercing phase. In another aspect, the target displacement profile 450 may control multiple, sequential injections each having a biphasic profile with a piercing phase and a drug delivery phase. In practice, the actual displacement profile of the plunger 120 may vary from the ideal target displacement profile according to physical limits of the system and other constraints.

Both the measured displacement value x(t) and the target displacement value x_{T}(t) are provided to the summing block 452. The summing block 452 subtracts the measured displacement value x(t) from the target displacement value x_{T}(t) to obtain an error signal x_{E}(t). The error signal x_{E}(t) is provided to the motor control signal generator 454 which converts the error signal to a motor control signal y(t). The motor control signal y(t) is provided to the three-phase motor controller 141 or other suitable drive system, which in turn drives the motor 126 according to the motor control signal y(t).

In some examples, the rotary motor 126 may be a three-phase motor with three windings 447 and three Hall sensors 449, each Hall sensor 449 corresponding to a different one of the three windings 447. Each of the windings 447 is wrapped around a laminated soft iron magnetic core (not shown) so as to form magnetic poles when energized with current. Each of the three Hall sensors 449 generates a corresponding output signal 456 in response to presence (or lack of) a magnetic field in its corresponding winding 447.

The three-phase motor controller 141 includes a switch control module 445 and a switching module 448. The switching module 448 includes three pairs of switches 451 (with six switches 451 in total), each pair of switches corresponding to a different one of the windings 447 of the rotary motor 126 and configurable to place the corresponding winding 447 into electrical connection with the power supply 143 (whereby the winding is energized) or with ground. The switch control module 445 receives the motor control signal y(t) from the injection controller 135 and the three Hall sensor output signals 456 as inputs and processes the inputs to generate six switch control signals 455, each switch control signal 455 configured to either open or close a corresponding switch 451 of the switching module 448.

The above-described configuration implements a feedback control approach to ensure that a combination of the controlled torque applied to the screw 332 of the ball screw 130 due to the motor 126 causes the displacement of the plunger to track the target displacement profile 450 as the screw 332 is displaced.

### Power Supply

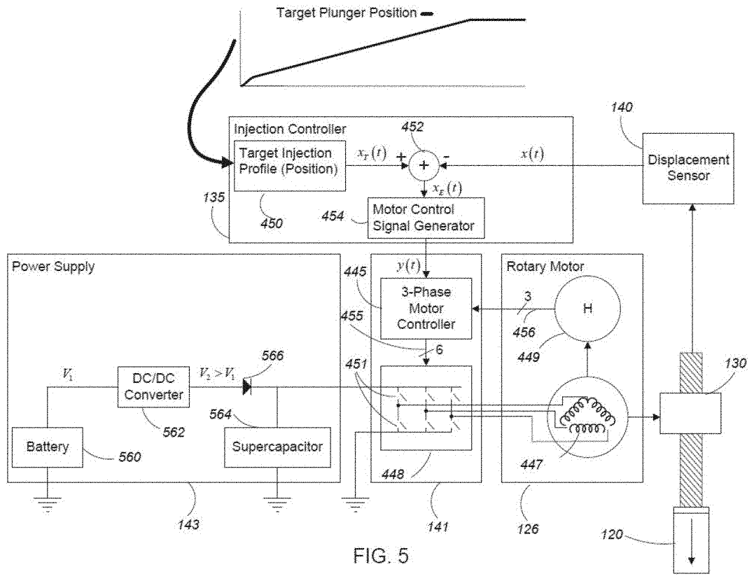

Referring to FIG. 5, in some examples, the power supply includes a battery 560 (e.g., a Nickel Cadmium battery, a Nickel-Metal Hydride battery, a Lithium ion battery, an alkaline battery, or any other suitable battery type) configured to supply a voltage V to a DC/DC converter 562 (e.g., a boost converter). The DC/DC converter 562 receives the supply voltage V from the battery 560 as input and generates an output voltage V₂ greater than V₁. In some examples, the DC/DC converter 562 is configured to boost the supply voltage by a factor in the range of 5 to 20. While the battery 560 may be rechargeable, the battery 560 may also usefully store sufficient energy for multiple injections, such as two or more one milliliter injections, e.g., from replaceable single-dose cartridges or from a single, multi-dose cartridge.

The output voltage V₂ may be provided in parallel to a supercapacitor 564 and to the switching module 448 of the three-phase motor controller 141 via a diode 566. In operation, the output voltage V₂ charges the supercapacitor 564 while the transdermal injection device 100 is inactive. When an injection operation commences, the switches 451 of the switching module 448 close (according to the switch control signals 455), connecting the windings 447 of the rotary motor 126 to the supercapacitor 564. This results in a discharge of the supercapacitor 564, causing current to flow through the windings 447 of the rotary motor 126 and induce rotation of the rotary motor 126.

In some examples, the supercapacitor 564 includes a number of supercapacitors coupled together with a switching network. When the transdermal injection device 100 is inactive, the switching network may be configured so that the number of supercapacitors is connected in parallel for charging. When an injection is initiated, the switching network may be reconfigured so that the number of supercapacitors are serially connected for discharge. In some examples, the supercapacitor 564 is configured to deliver a peak power of 200 Watts or more to the ball screw 130 via the rotary motor 126.

In general, the supercapacitor may be any high-capacity capacitor suitable for accepting and delivering charge more quickly than a battery or other source of electrical energy. A wide variety of supercapacitor designs are known in the art and may be adapted for use as the supercapacitor 564 contemplated herein, such as double-layer capacitors, pseudocapacitors, and hybrid capacitors. Similarly, the supercapacitor 564 may usefully include any number and arrangement of supercapacitors suitable for delivering electrical power in an amount and at a rate suitable for driving a rotary motor 126 of an injection device 100 as contemplated herein.

### Target Displacement Profile

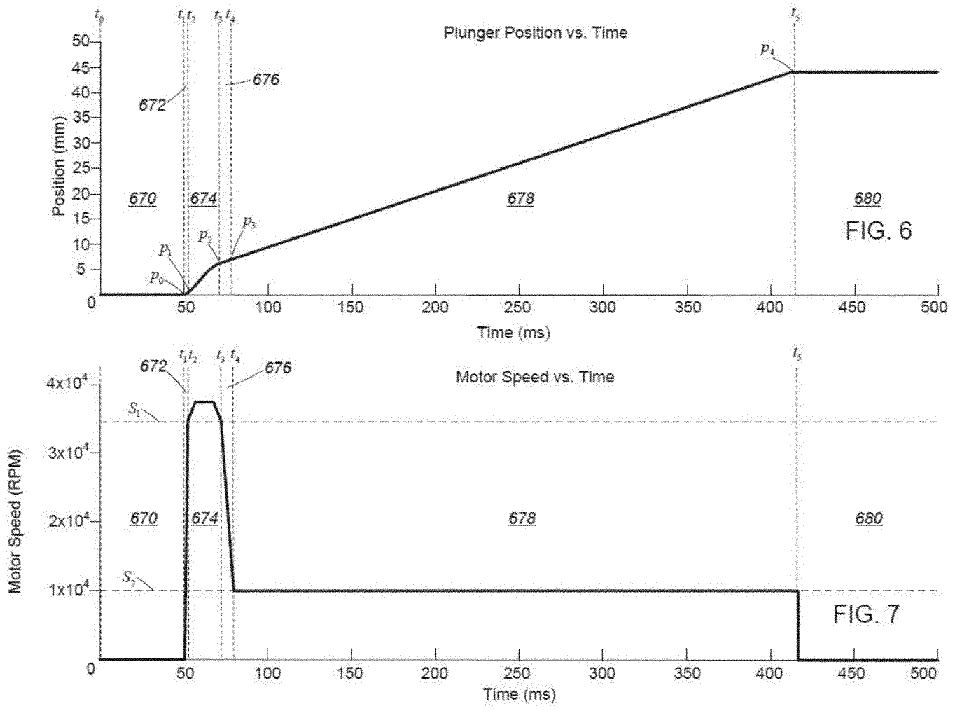

Referring to FIG. 6, one example of a target displacement profile includes a number of injection phases, each associated with a corresponding time interval. A first injection phase 670 is associated with a first time interval extending from time to to time t₁. In the first injection phase 670, the target displacement of the plunger 120 is at a constant initial position p₀ where the plunger 120 is engaged with the injectate in the chamber 106. In this phase, the injection device 100 is generally prepared to perform an injection operation. In general, the first injection phase 670 may be preceded by any number of preparatory steps or phases, such as loading of an injectate (or a cartridge containing an injected) into the injection device, the removal of bubbles from the injectate as necessary or appropriate, measuring environmental conditions, measuring parameters of an injection site, and any other steps or combination of steps useful for performing, or preparing to perform, a needle-free injection as contemplated herein. In one aspect, the rotary motor 126 may be mechanically engaged with the ball screw actuator 130 (or any other suitable linear actuator) while the rotary motor 126 is stationary in the first injection phase 670. That is, the rotary motor 126 may be pre-engaged with the ball screw actuator 130 and preload to remove any mechanical slack in the mechanical components of the system. In this configuration, a mechanical switch or the like may be used to prevent relative movement of the components, and/or a gate or seal may be used at the nozzle exit to prevent leakage of drug from the chamber 106. In another aspect, the rotary motor 126 may be slightly spaced apart from engagement with the ball screw actuator 130. In this latter configuration, the rotary motor 126 may usefully accelerate (while unloaded) into engagement with the ball screw actuator 130 at an end of the first injection phase 670 or at a beginning of the second injection phase 672 to facilitate a greater initial velocity of injectate from the nozzle. This may, for example, include a single rotation of the rotary motor 126 from engagement with the ball screw actuator 130, or a fractional rotation suitable to facilitate very high initial rotational acceleration.

A second injection phase 672 is associated with a second time interval extending from time t₁ to t₂. In the second injection phase 672, movement of the plunger 120 may be initiated. In this phase, the target displacement of the plunger 120 increases at a relatively high first rate to move the plunger 120 from the initial position p₀ to a first position p₁. In general, the motion of the plunger 120 in this phase may cause a jet of injectate to be ejected from the chamber 106 of the injector head 104 (via the opening 114) with a first velocity V₁ at least sufficient to pierce human tissue to a subcutaneous depth. In some examples, the second injection phase 672 spans a time interval less than 100 ms (i.e., the difference between t₁ and t₂ is less than 100 ms). In some examples, the second injection phase 672 spans a time interval less than 60 ms (i.e., the difference between t₁ and t₂ is less than 60 ms). In some examples, the second injection phase 672 spans a time interval less than 10 ms (i.e., the difference between t₁ and t₂ is less than 10 ms).

More generally, the injection device 100 may be configured so that in this second injection phase 672, the plunger 670 transitions from a stationary position to the target velocity at a sufficient rate for the initial stream of injectate to achieve a piercing velocity substantially instantaneously, e.g., without substantial leakage or loss of injectate at the surface. By configuring the linear drive system described above to accelerate in this manner from a fixed position to a piercing velocity, the injection device 100 may advantageously mitigate loss of injectate. As a further advantage, an injection device with this capability can usefully perform multiple sequential injections without requiring any physical recharge or resetting of a mechanical stored energy system.

A third injection phase 674 is associated with a third time interval extending from time t₂ to t₃. In the third injection phase 674 the target displacement of the plunger increases at a rate substantially the same as the first rate to move the plunger 120 from the first position p₁ to the second position p₂. In this third injection phase 674, the plunger 120 may be moved at a rate to cause the jet of injectate to be ejected from the chamber 106 of the injector head 104 with a second velocity V₂ greater than or equal to the first velocity V₁. While the rate of plunger 120 movement and the velocity of the injectate stream may vary within this third injection phase 674, e.g., according to limitations on control precision, physical system components, and so forth, the plunger 120 should generally be driven at a minimum velocity suitable for piercing tissue at a target site to a desired depth for delivery of the injectate. The jet of injectate may also have a maximum velocity selected to avoid over-penetration or other undesirable tissue damage.

A fourth injection phase 676 is associated with a fourth time interval extending from time t₃ to time t₄. In the fourth injection phase 676 the target displacement of the plunger 120 increases at a third rate, relatively slower than the first rate, to move the plunger 120 from the third position p₃ to a fourth position p₄. In this fourth injection 676, the injection device 100 may generally decelerate the plunger 120 to cause the jet of injectate to eject from the chamber 106 of the injector head 104 with a third velocity V₃ less than the first velocity V₁, which may generally be any velocity suitable for non-piercing delivery of additional injectate at a current depth of the stream of injectate within the target tissue.

A fifth injection phase 678 is associated with a fifth time interval extending from time t₄ to t₅. In the fifth injection phase 678 the target displacement of the plunger 120 continues to increase at the third rate to move the plunger 120 from the fourth position p₄ to the fifth position p₅. In the fifth injection phase 678, the injection device 100 may generally deliver the injectate—typically a majority of the injectate in the chamber 106—at a subcutaneous depth achieved during the prior, piercing phase. The rate of movement may be generally constant, or may otherwise vary consistent with maintaining subcutaneous drug delivery without further piercing of the tissue.

It will be appreciated that some continued piercing may occur during the fifth injection phase 678. Provided that any additional piercing does not create a pathway below subcutaneous depth within the target tissue that might result in loss or misdelivery of therapeutic dosage, then this additional piercing will not affect the efficacy of transdermal drug delivery. It will also be understood that the total displacement of the plunger 120 will control the volume of drug delivered over the course of an injection, and a duration of the fifth injection phase 678 may correspondingly be selected according to an intended dosage.

Finally, a sixth injection phase occurs after time t₅. In the sixth injection phase the target displacement of the plunger 120 stops increasing, substantially halting the plunger 120 at a sixth position p₆. The sixth injection phase is associated with completion of the injection operation. As noted above, from this position, additional injection cycles may be initiated, provided of course that sufficient additional drug remains in the injection device 100 for completing additional injections.

In order to quickly achieve a piercing velocity and avoid loss of drug at the surface of an injection site, the second injection phase 672 (where acceleration of the injectate occurs) may be short relative to the piercing phase that is maintained once the piercing velocity is achieved. Thus, in some examples, the time interval associated with the third injection phase 674 is in a range of two to twenty times as long as the time interval associated with the second injection phase 672. In some examples, the time interval associated with the second injection phase 672 has a duration between 30 milliseconds and 100 milliseconds and the time interval associated with the third injection phase 674 has a duration between 100 milliseconds and 1000 milliseconds. More generally, the duration of each phase may depend on the diameter of the injectate stream, the properties of the injectate, the characteristics of the tissue at the injection site and so forth. Thus, the injection profile may usefully employ any durations suitable for accelerating to a piercing velocity sufficiently rapidly to avoid substantial loss of injectate, maintaining a piercing velocity until a target depth (e.g., subcutaneous depth) is achieved, and then maintaining a non-piercing velocity to deliver a full dose at the target depth.

It will also be understood that, while a single injection cycle is illustrated, the injection device 100 contemplated herein may usefully be configured for multiple, sequential injections. As such any number of injection cycles might usefully be performed, and any such multi-injection applications are expressly contemplated by this description.

### Rotary Motor Speed

Referring to FIG. 7, in the first injection phase 670, the injection controller 135 controls the rotary motor 126 to maintain its speed at substantially 0 rotations per minute (RPM) to ensure that the plunger 120 remains stationary at the initial position p₀. This may include actively maintaining the rotary motor 126 in a fixed position, e.g., by monitoring the position and activation the rotary motor 126 in counter-response to any detected motion or drift, or by control a magnetic, mechanical, or electromechanical lock that securely engages the plunger 120 in the initial position p₀. In another aspect, this may include passively maintaining the rotary motor 126 in the fixed position by withholding control signals or drive signals from the rotary motor 126. It will also be understood that combinations of the foregoing may advantageously be employed. For example, the plunger 120 may be locked with a mechanical lock during storage or while otherwise not in use, and then the rotary motor 126 may be used to electromechanically and actively lock the position of the plunger 120 when the mechanical lock is disengaged to prepare for an injection. In this manner, power may be conserved during long term storage, while the position can be securely and controllably locked using the rotary motor 126 in an interval immediately prior to injection in order to prevent, e.g., leakage of an injectate.

In the second injection phase 672, the injection controller 135 may control the rotary motor to accelerate from 0 RPM to a first rotary motor speed S₁ (e.g., 33,000 RPM), causing the plunger 120 to move from the initial position p₀ to the first position p₁. In the third injection phase 674, the injection controller 135 may control the rotary motor 126 to maintain a speed at or above the first rotary motor speed S₁ causing the plunger 120 to move from the first position p₁ to the second position p₂. In the fourth injection phase 676, the injection controller 135 may control the rotary motor 126 to decelerate to a second rotary motor speed S₂ (e.g., 11,000 RPM) less than the first rotary motor speed S₁, causing the plunger 120 to move from the second position p₂ to a third position p₃. In the fifth injection phase 678, the injection controller 135 may control the rotary motor 126 to maintain the second rotary motor speed S₂, causing the plunger 120 to move from the third position p₃ to a fourth position p₄ at a substantially consistent rate for delivery of an injectate at a target depth for an injection.

In the sixth injection phase, the injection controller 135 may control the rotary motor 126 to decelerate its speed from the second rotary motor speed S₂ to RPM, causing movement of the plunger 120 to substantially halt at the fourth position p₄.

While the supercapacitor 564 in the power supply 143 described above may be used during any portion of the injection delivery, the supercapacitor 564 may be particularly advantageous where high mechanical loads are anticipated, e.g., during the initial acceleration and piercing phases, as well as where necessary or helpful to quickly decelerate or stop the plunger 120, e.g., at the fourth position p₄. Thus, the supercapacitor 564 may be specifically used during the second injection phase 672, the third injection phase 674, and optionally the fourth injection phase 676 if high power is required to maintain a target speed even during a deceleration of the injectate to a drug delivery velocity, and/or if high power is required to quickly decelerate or stop the plunger 120.

### Injectate Velocity

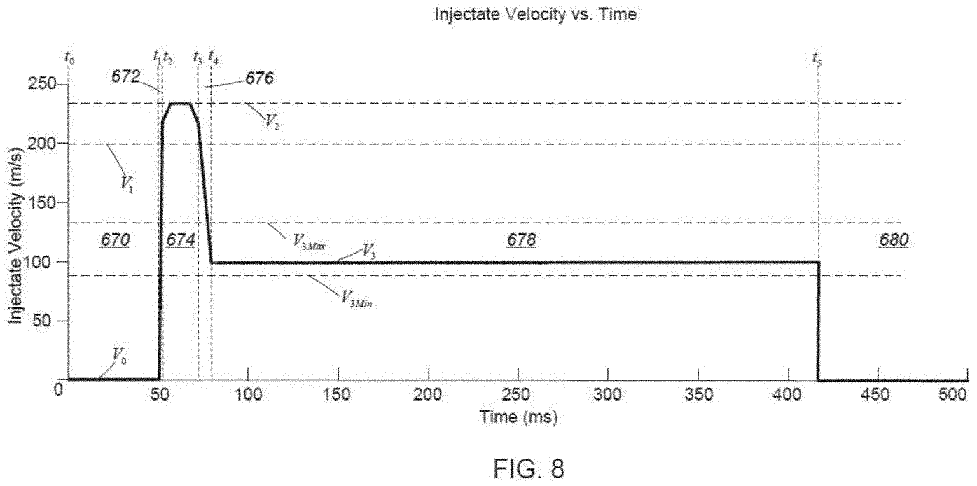

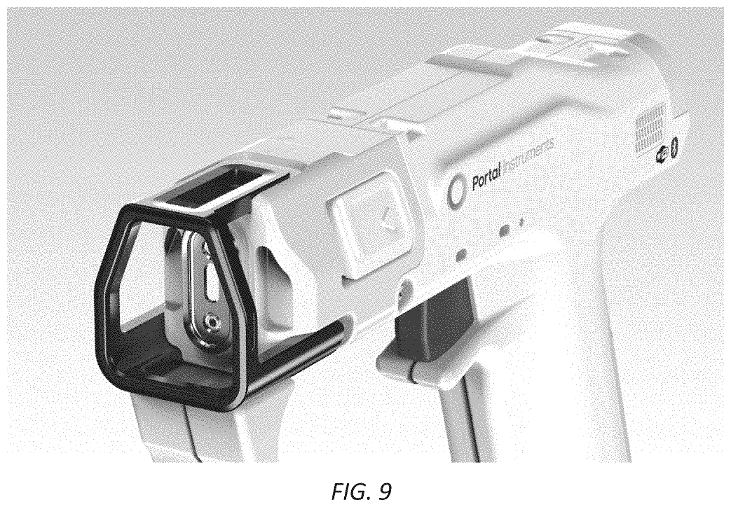

Referring to FIG. 8, in the first injection phase 670, no injectate is ejected from the chamber 106 (i.e., the initial injectate velocity, V₀ is 0 m/s). In the second injection phase 672, the injectate velocity increases from 0 m/s to the first velocity, V at least sufficient to pierce human tissue. In some examples, the first velocity V₁ is at least 200 m/s. If piercing is not initiated quickly, then there may be substantial loss or leakage of drug. Thus, in some embodiments, the rotary motor 126 may usefully be configured to reach the first velocity V₁ for injection from a stationary starting point in not more than three rotations, such as less than two rotations, or less than one rotation.

In the third injection phase 674, the injectate velocity may be maintained at a second velocity V₂ greater than or equal to the first velocity V₁ in order to continue piercing tissue at a target site. Where the first velocity V₁ is a minimum velocity for piercing tissue, then the second velocity V₂ is preferably maintained above the first velocity V₁ in order to continue piercing throughout the third injection phase 674. However, the first velocity V₁ may instead be a minimum velocity or an optimum velocity to initiate piercing, in which case the second velocity V₂ may usefully be any velocity greater than, equal to, or less than the first velocity V₁ suitable for continuing to pierce tissue to the desired, target depth. Similarly, the second velocity V₂ may vary over the duration of the third injection phase 674 provided that the second velocity V₂ remains within this window of useful piercing velocities.

In the fourth injection phase 676, the injectate velocity may decreases to a third velocity V₃ (in a range between a maximum third velocity V_{3Max} and a minimum third velocity V_{3Min}) sufficient to deliver the majority of the injectate in the chamber 106 at a subcutaneous depth. In the fifth injection phase 678, the injectate velocity may be substantially maintained at the third velocity V₃ while the majority of the injectate in the chamber 106 is delivered to the subcutaneous depth through the channel created during the third injection phase 674. It will be appreciated that the third velocity V₃ may vary over the course of the fifth injection phase 678 between any values—typically greater than zero and less than a piercing velocity—consistent with delivery of the injectate at the target depth. Finally, in the sixth injection phase 680, the injectate velocity may decrease to 0 m/s as the injection operation completes.

### Injectate

In some examples, the volume of injectate in the chamber is at least one milliliter. Thus, in one aspect the injection device 100 may be configured to deliver one milliliter of drug subcutaneously in a single dose, or as a number of sequential doses over time, e.g., to different locations or over the course of an extended dosing schedule. Where a large number of sequential doses are intended, or where a larger single dose is intended (e.g., more than one milliliter) the chamber may usefully have a greater volume. For multi-dose applications, the contents of the chamber 106 may be conveniently distributed in discrete doses using a rotary motor and linear drive system as contemplated herein. In some examples, the volume of injectate in the chamber is less than or equal to approximately 0.5 milliliters. In some examples, the volume of injectate in the chamber is less than or equal to approximately 0.3 milliliters. In some examples, the volume of injectate in the chamber is a therapeutic amount of injectate.

In some examples, the injectate includes a biological drug. In some examples, the injectate has a viscosity of at least three centipoise at a temperature between two degrees and twenty degrees Celsius. In some examples, the injectate has a viscosity of about three centipoise to about two hundred centipoise at a temperature between two degrees and twenty degrees Celsius. Thus, the system described herein may usefully be employed with large molecule therapeutics or other drugs having relatively high viscosities.

### MISCELLANEOUS

In one aspect, the injection controller may be configured to cause the needle-free transdermal injection device 100 to perform a number of sequential injection operations in close temporal proximity to one another. The injection device 100 may usefully be instrumented to support this operation by sensing movement of the injection device 100 and providing tactile, visible, audible or other feedback to aid in navigating the user through a multi-injection procedure.

In another aspect, a number of sequential injection operations may be performed without having to reverse the movement of the rotary motor (i.e., to withdraw the plunger). Thus, where additional injectate remains in the injection device 100 at the end of an injection cycle sufficient for an additional dose, the rotary motor 126 may remain stationary, and a second, complete injection cycle may be initiated from this new starting position. In this context, the rotary motor 126 may be manually locked, or electromagnetically maintained in a fixed location in order to prevent leakage or other loss of therapeutic product.

In some examples, the linkage (e.g., the ball screw linkage) is bidirectionally coupled to the rotary motor and the plunger such that bidirectional displacement of contents in the chamber is possible, e.g. by moving the plunger toward an exit nozzle to eject contents, or moving the plunger away from the exit nozzle to load additional drug into the injection device 100.

In some examples, the transdermal injection device includes a sensor system for detecting when the device is properly positioned for performing an injection operation. In some examples, once the device is properly positioned, the injection controller is configured to initiate the injection operation without any observable latency. That is, the sensor system may monitor the injection device 100, determine when the injection device 100 is properly positioned and stationary, and then initiate an injection. Depending on the duration and feel of the injection, the injection may usefully be preceded by a beep, vibration, or other human-perceptible signal alerting a user that the injection is about to occur.

In some examples, one or more conventional capacitors (e.g., electrolytic capacitors) can be used instead of the supercapacitor.

In some examples the injection controller is configured to prevent two or more injection operations within a predetermined minimum injection cycle time. Thus, for example, where a dosing regimen specifies a minimum time before injections, or where an injection is being delivered as a sequence of injections in different but adjacent locations, the injection controller may monitor activation of the injection device 100 to ensure that any rules for a corresponding injection protocol are adhered to.

In some examples, the needle-free transdermal injector head is formed as a removable cartridge for containing injectate. The removable cartridge has an opening with a predetermined shape for ejecting the injectate in a stream with a predetermined shape. In some examples, the needle-free transdermal injector includes a movable cartridge door mechanism. A user can interact with the movable cartridge door mechanism to load cartridges into the needle-free transdermal injector and to unload cartridges from the needle-free transdermal injector.

While the above description relates primarily to methods and apparatuses for the injection of therapeutics through human tissue to a subcutaneous depth, it is noted that, in some examples the methods and apparatuses described above are used for injection of therapeutics through human tissue to other shallower or deeper depths. For example, the methods and apparatuses can be used for a shallow injection of therapeutics into the dermis, or for a deeper injection though the subcutaneous layer of fat and connective tissue into a patient's musculature.

### Mass Injections

This disclosure relates to an injection device, such as a handheld, needle-free transdermal injection device, for relatively large-scale applications including mass inoculations or livestock drug delivery.

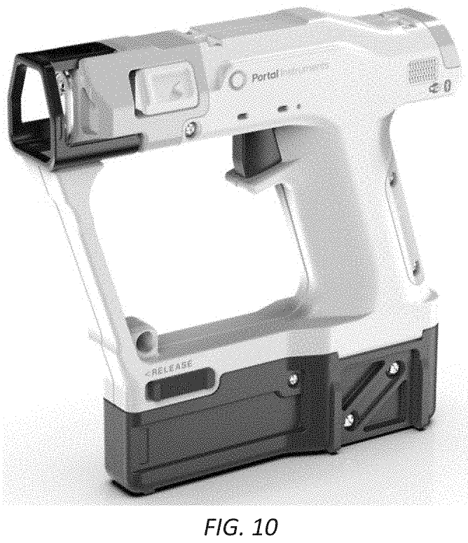

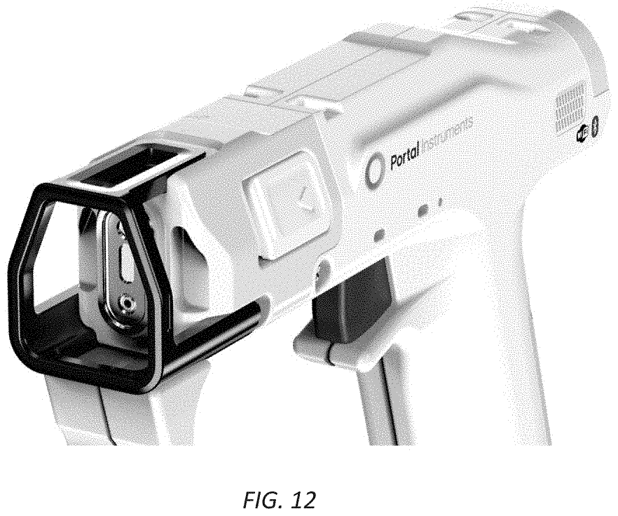

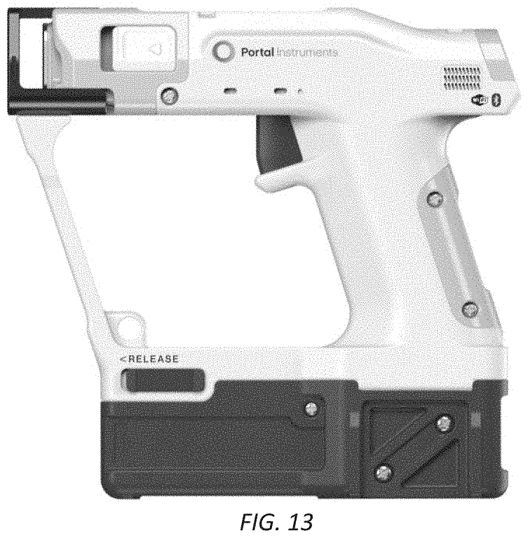

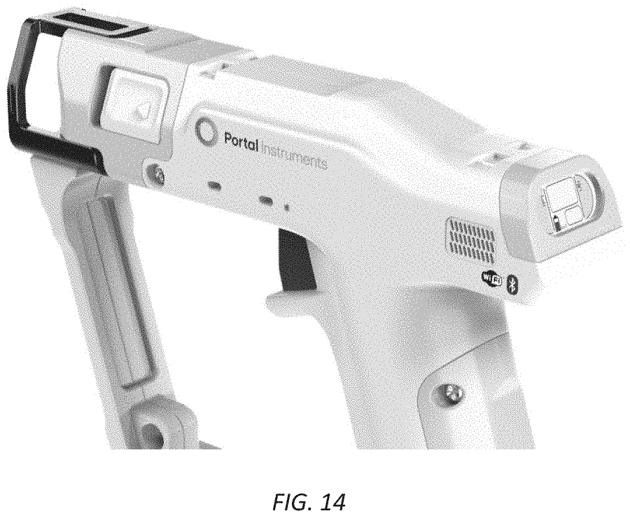

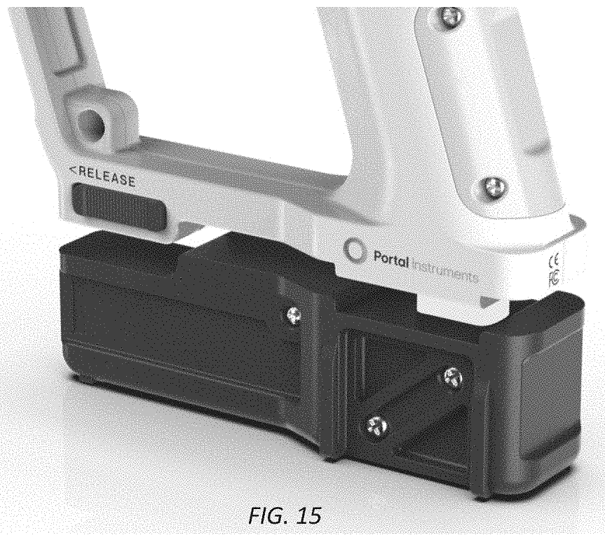

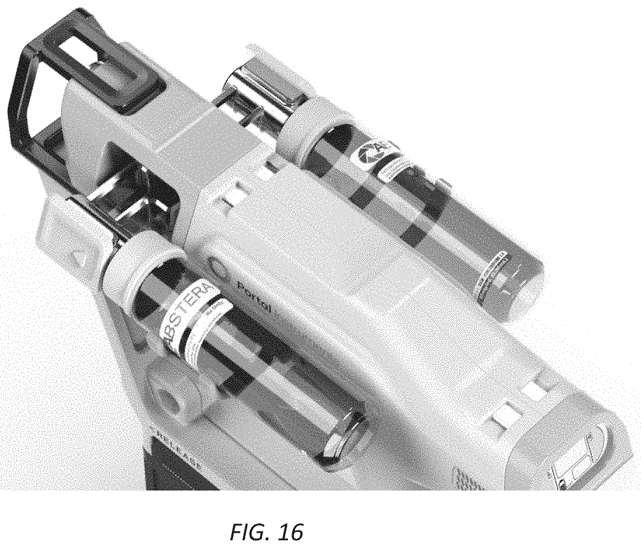

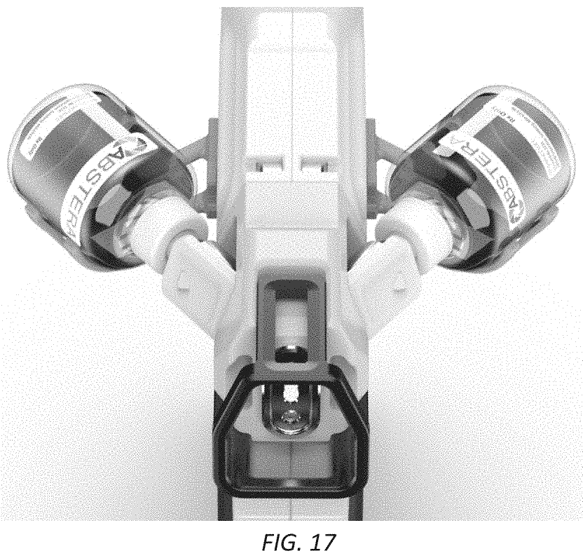

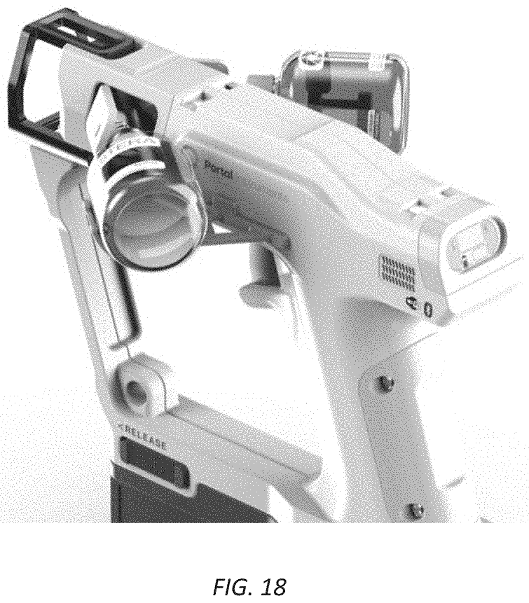

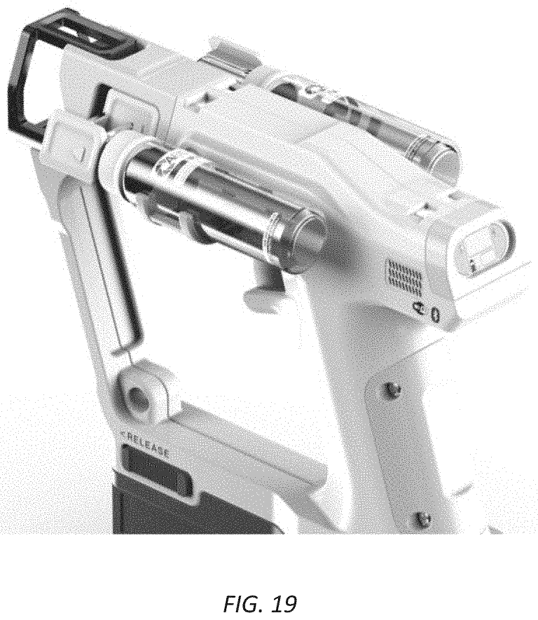

An injection device may utilize any of the features described above with reference to FIGS. 1-8. In general, the injection device may be handheld, such that an operator can inject a plurality of humans or animals in sequence. For example, an operator may be able to inject a number of animals (e.g., cows, pigs, chickens, goats, sheep, and so on) using an injection device of the present disclosure. This may be accomplished through the incorporation of various useful features, e.g., including but not limited to multiple barrels, multiple cartridges to store one or more drugs, adaptations of actuation modes to the agricultural context, improvements to power supplies and systems, modularity, and so on. FIGS. 9-19 show exterior perspective views of needle-free injection devices for mass inoculation of the present disclosure.

### Reservoirs

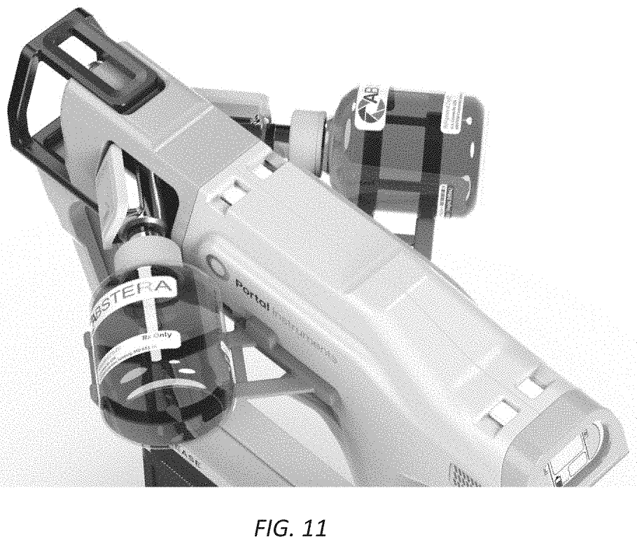

As shown in FIGS. 11 and 16-19, an injection device may include more than one container (e.g., two containers) for drugs to be injected into animals, where these containers may be referred to herein as “reservoirs.” The plurality of reservoirs associated with an injection device may include the same drug e.g., for redundancy or increased capacity. The plurality of reservoirs may also or instead include different drugs. In this manner, the same injection device may be used for treatments involving multiple drugs, supplements and the like, e.g., the injection of two different drugs into the same subject. This embodiment can also support the selection among two or more different drugs for two different subjects (e.g., animals that are a different species or animals having a different age, sex, weight, health status, and so on).

Also, or instead, the plurality of reservoirs may include portions of a drug that can be mixed before injection, e.g., within the injection device. For example, each cartridge may feed into the same barrel or actuating mechanism for mixing and subsequent injection into human or animal. Alternatively, the plurality of reservoirs may include different drugs to be administered to the same human or animal at substantially the same time (e.g., without prior mixing), such as by using two, separate jetted streams of the drug for injection through two distinct apertures in the injection device. Further still, the plurality of reservoirs may include different drugs to be administered sequentially to an animal. Each drug may be associated with its own actuator, or the drugs may share an actuator. Each drug may be injected through its own aperture or nozzle at the end of the barrel of the injection device, or each drug may share an aperture for release from the injection device during injections.

The reservoirs may be sized and shaped, and otherwise structurally configured, for relatively easy loading and unloading onto the injection device, e.g., before and after use. For example, the reservoirs may be connectable to the needle-free injector using a fitting or a coupling that can connect without tools, such as a clamp, screw and/or helical threads, bayonet, Luer lock, twist-lock, or other suitable connectors. In another aspect, the reservoirs may be separate from a handheld injector, e.g., in a backpack or coupled to a belt or shoulder straps, and connected to the handheld hardware through tubes or the like.

The reservoirs may be configured to display the contents therein. For example, the reservoirs may include a label or a display that shows information regarding the contents of the cartridge, such as a name of a drug, an expiration date, and so on. The reservoirs may also or instead be configured to display other information including without limitation an amount of contents remaining in the cartridge, how many injections have been administered, and so on.

The internal volume of the reservoirs may include a positive pressure therein. For example, a reservoir may include a plurality of cannulas, where at least one of these cannulas is structurally configured for fluid communication into the injection device and where another cannula is accessible from an external environment for pressurization, refilling, and the like. More specifically, a reservoir may include an extra cannula into the reservoir itself, where the extra cannula can be used to push air, gas, or another fluid into the reservoir to create a positive pressure within the internal volume to support an injection process, e.g., by facilitating dispensing a dose of the drug into a pre-injection chamber.

### Actuator

### Single Actuator

The injection device may feature a single actuator, e.g., even in embodiments that feature a dual barrel or a plurality of reservoirs for a plurality of drugs as described above. Stated otherwise, each drug in an injection device may be driven by the same actuator-in series or in parallel. The actuator may feature a single drive that is in communication with each reservoir or chamber that stores or otherwise contains or transports the drugs for the injection device, and the actuator may power injections from multiple barrels in a single linear motion, e.g., by switching from a coupling to a first chamber to a coupling to a second chamber during a single linear motion, or the actuator may use multiple linear strokes, each driving a different injection from a different chamber.

The injector may automatically dose from multiple drug sources, or a user may be provided with a switch or other control that permits the selection of one or more different drugs that are available, and/or that permits manual mixing (e.g., by volume) of multiple drugs within the injector reservoirs.

The reservoirs may be of any practical volume, shape, and materials construction to provide for the necessary amount of injectate to be dispensed while maintaining maneuverability of the needle-free injector. This disclosure is not limited to the construction and size of the fluid reservoirs. In some embodiments, the reservoir may be a reservoir fluidly coupled to the housing by a flexible conduit. For example, a reservoir coupled to the housing by a flexible conduit may be a wearable reservoir capable of holding a larger volume of an injectate, such as a backpack-style reservoir or the like. Larger reservoirs may be gravity-fed or siphon-fed. A wearable reservoir may connect to the housing at the same location where a bottle-style reservoir connects to the needle-free injector using a suitable connector of sufficient length, such as a hose, that terminates in appropriate fluid connectors. A flexible connection may also include appropriate strain relief, such as overmoulding, at the connections to ensure repeated connect-disconnect cycles without leaking. In further embodiments, a reservoir may be a floor-based reservoir, such as a tank on a movable or rolling cart, to permit substantially larger volumes of injectates to be used that exceeds a weight that a user or operator may comfortably wear or carry. The connection between a floor-based fluid reservoir and the needle-free injector may be similar to the connection between the needle-free injector and a wearable reservoir, such as a flexible hose with appropriate fluid-tight connectors at its termini, such as a clamp, screw and/or helical threads, bayonet, Luer lock, twist-lock, or other suitable connectors.

### Multiple Actuators

The injection device may feature a plurality of actuators, e.g., a dual actuator. For example, two or more actuators may be driven by the same power system or power source in an implementation.

An actuator may include one or more springs, gas supplies, or other electromechanical systems to augment operation of the injector. In general, an unregulated power source such as a spring, pneumatic pulse, or combustion event, may be used to provide partial power to the injector in an open loop supply, and a rotary motor system such as any of the rotary systems described herein may be used to increase or decrease the axial force applied to an injection mechanism in order to achieve a desired injection profile in a closed loop that drives the injector toward a target velocity (or other control variable).

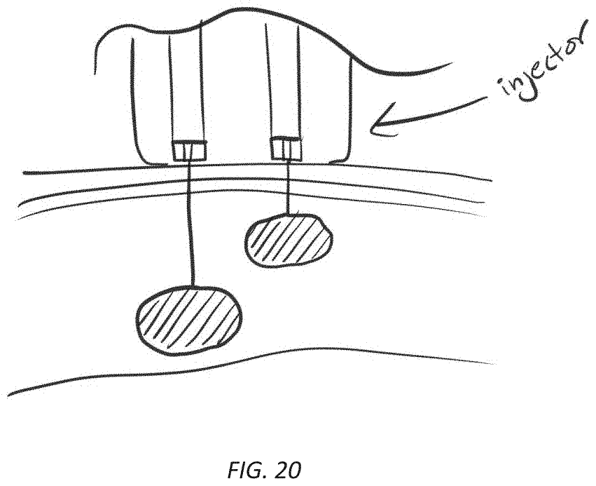

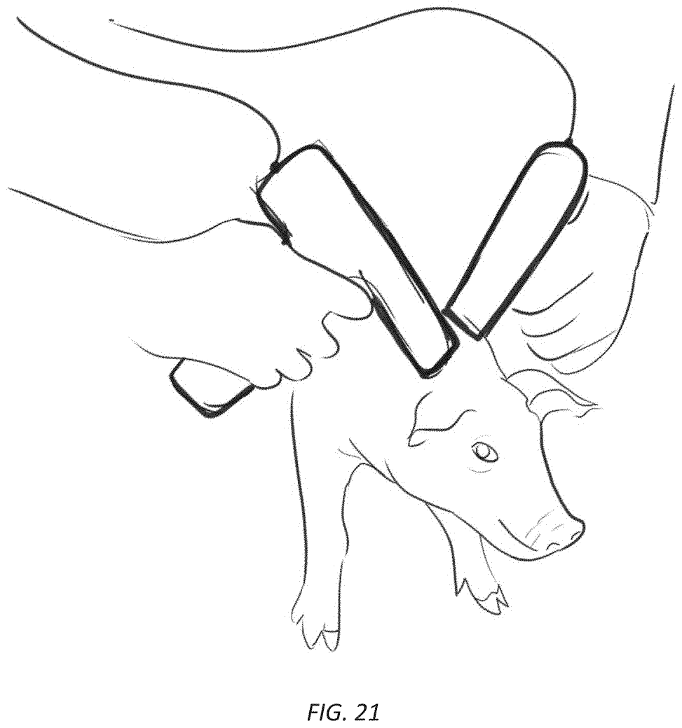

An illustration of an injection process using a dual actuator needle-free injector is illustrated in FIG. 20. In the configuration of FIG. 20, the actuators are parallel and the injectate velocity is such that the injectates are delivered to different depths within the tissue of the subject. In some embodiments, dual actuators may comprise separate needle-free injectors, such as illustrated in FIG. 21. With reference to FIG. 21, the dual actuators may be positioned at an angle such that the injectate can be delivered to parallel tissue locations on a subject, such as both sides of a neck of a pig as illustrated. In this configuration, the chambers may be separated so an end user or operator can hold one in each hand. Actuation of the plunger may be achieved on one needle-free injector to activate both injection. Alternatively, separate actuation of each needle-free injector may be required for safety purposes.

### Power Source and Power System

The injection device may feature a relatively large-capacity battery that can be used for multiple injections—e.g., about 100 or more injections. The battery may supply direct, instantaneous power to an injection device. With a suitable battery (e.g., supplying adequate current and voltage), the capacitors may be omitted from a power circuit for the rotary drive system.

### Interlock

The injection device may include an interlock, e.g., disposed on the front (distal) end of the device. The interlock may include a protective structure or cage on the front of the device. In one aspect, the interlock may trigger an injection upon contact with a target surface. The interlock may also or instead respond to contact with a target surface by unlocking the injection device for a user to otherwise actuate or trigger the injection.

### Sighting

The injection device may include one or more features for sighting a site for an injection. For example, the injection device may include at least one light source (e.g., an LED light or the like) that can be used for aiming and sighting the injection device for accuracy when performing an injection—e.g., by placing a red dot or the like on an injection site prior to injection. The sighting feature may also or instead include a laser or the like. An optical sight may also provide additional user feedback. For example, by detecting a particular animal, e.g., with a tag reader or the like as described below, the injector may first determine whether the animal has received an injection. If the current target has received an injection, the injector may project with a red LED, or project an “X” or other symbol or indicator that no injection should be made (this may also or instead be displayed on a user interface of the injector). If the current target has not received an injection, the injector may project with a green LED, or project an “O” or other symbol distinguishable from the no-injection symbol.

### Modularity

The injection device may include one or more features for modularity. That is, the injection device may include one or more modular components that can be removed and replaced with relative ease.

For example, in certain implementations, components on the injection device that touch the cartridges, drugs, or target surfaces, may be removable and replaceable, e.g., for cleaning and re-use.

In certain implementations, the injection device may support a disposable use product cycle. To this end, counters for certain components such as the interlock may be included to ensure that a certain component is used only a predetermined number of times.

### Injection Confirmation

The injection device may include one or more features for indicating or otherwise confirming that an injection (e.g., a successful injection) has taken place. For example, an injection confirmation feature may include marking an injection site after an injection. This may include automatically adding a dot of paint or another visible marking or identifier to an injection site. Thus, in one aspect, the injector may also include a paint or dye source, along with a delivery system that applies a mark to a target animal after an injection is complete.

### Detectable Markers

In general, animals for injection may be tagged with a detectable marker. For example, livestock may include electromagnetic emitters, e.g., RFID (or similar) data tags, near field communication transceivers, a fiducial marker, e.g., a Quick Response (QR) code, printed to a polymer tag, or the like on their ears or another feature of their anatomy, where these tags uniquely identify an animal for purposes of tracking and retrieval of corresponding information, or where the tags directly encode information regarding the animal, e.g., data related to an injection status of the animal, or some combination of these. The injection device may include one or more communication features that read from or write to such data tags. For example, the injection device may include a sensor, such as an optical sensor, a tag reader or the like, that is configured to acquire a signal from a detectable marker coupled or connected to the subject.

It will be understood that sensors, for example, tag readers, are available in passive and active forms, each with attendant advantages and disadvantages. One concern in the context of, e.g., a crowded pig sty, is the possibility that active tags, with a relatively greater communication range, might interfere with one another. Thus, where the underlying tag technology supports a range presenting possible signal overlaps, the tags may be explicitly tuned for a shorter range in order to ensure that the injector only reads information from a target of interest.

The sensor may read data from a tag and use the data to associate an injection with a specific animal or a specific animal species. In this manner, a user may learn whether a specific animal has received an injection or whether the specific animal requires an injection. Also, or instead, data may be transmitted to the detectable marker from the injection device, e.g., to indicate that an animal has received, or is about to receive, an injection.

The injection device may include one or more display features that work with the sensor and/or detectable marker for communicating an injection status of an animal. By way of example, the injection device may include a LED or another light source associated with its sensor. When a detectable marker is activated or successfully read, a visual indicator (e.g., the LED light) may create a visual indication of the injection status for the animal associated with the tag.

Additionally, or alternatively, a LED or other light source or visual marking may be included on a detectable marker itself, e.g., where the detectable marker may be actuatable to display the light to indicate an injection status. By way of example, one or more colored lights may illuminate on a detectable marker to indicate an injection status, such as a red light for an animal that should not receive an injection (e.g., when the animal has previously received the injection) and a green light for an animal that should receive an injection (e.g., when the animal has not previously received the injection). Also, or instead, a detectable marker may display an indicator on the injection device itself, or the injection device may otherwise display such an indicator itself after reading the tag.

This type of tracking also facilitates other injection tracking applications. For example, where livestock is mixed, e.g., by periodic merging into larger groups and separation into smaller groups, a particular animal may be checked for previous treatments, and dosage may be administered from one or more available cartridges in a bespoke manner for that animal. This alleviates the need for physical tracking. Similarly, when a known collection of animals is inventoried in a database, the injector or a supporting computer system may provide alerts that one or more doses have not been administered. More generally, many types of tracking and dose management may usefully be deployed in a system that includes animal tags and an injector with a tag reader as generally described herein.

When an animal should not receive an injection (e.g., where this information is received by the injection device through a sensor and controller that receives and interprets data from a tag on the animal), the injection device may lock or otherwise prevent an injection from being administered to the animal. In such instances, the injection device may request an override by a user to unlock or otherwise re-enable the injection device for injections.

In certain implementations, the injection device may include a communications interface for transmitting data to a detectable marker of an animal, or for otherwise transmitting data (e.g., data read from a detectable marker). For example, an injection device may transmit information to the detectable marker including data such as the drug(s) that have been administered and other information regarding the drug(s), a date and time, dosing information, and so forth.

The detectable marker and/or the sensors may feature long or short-range communication devices and systems. For example, the detectable marker and/or the sensors may include active or passive RFID tags, near field communication transceivers, and so forth. On the other hand, a tag may include medium or long range communications capabilities such as Wi-Fi, cellular, and Bluetooth communication, or an infrastructure may leverage the capabilities of the injector to record data from the animal tags, and communicate with a remote system using such medium or long range communications. It will also be understood that many farms are in rural environments without reliable wireless communications infrastructure. In this context, the injector may record information from the animal tags, and may be used to port this information, either directly or through a connection to a remote resource, to a computer system with a suitable database for storing the animal tag information and/or correlating the information to particular animals.

The detectable marker may contain useful information regarding the animal, the injections, or the drugs. By way of example, the detectable markers may serve as an identifier that allows the injection device to determine what drug a particular animal should be injected with, at what dose, and so forth. In this manner, dosing or the like may be automatically adjusted based on information received from a detectable marker.

In some embodiments, the information acquired from the detectable marker may be used to operate the needle-free injector in one or more different modes of operation. For example, the detectable marker may be scanned by the sensor, and the acquired information may cause the needle-free injector to automatically inject the subject with the injectate, that is, without the intervention of the end user or operator. The automatic injection may occur, upon processing the information contained in the detectable marker, immediately or after a predetermined time delay. For example, a predetermined time delay may allow for an end user or operator to position and/or re-position the needle-free injector to the correct location on the subject if necessary. Alternatively, or in addition, the acquired information may be used inform the end user or operator, such as by an indication or alert on a graphical display, that the subject is eligible for an injection. In this configuration, the end user or operator actuates the needle-free injector, for example by squeezing a trigger, to allow the needle-free injector to deliver the injectate to the subject.

### Graphical Interface

The needle-free injector may include a graphical interface, such as a small LED display or the like, on the needle-free injector. The graphical display may be integrated into the housing and operatively coupled to the controller such that the graphical interface can present information pertaining to at least one of the status of the needle-free injector and an injection process. In one aspect, the graphical interface may face toward a user when the injector is being held for use, that is, the graphical interface is positioned near a rear portion of the housing. The graphical interface may be configured to display a status of the needle-free injector that includes at least one of a remaining charge on a battery and service messages for the needle-free injector. For example, the graphical display may provide a user an indication that the needle-free injector needs warranty service due to an operating parameter having a magnitude outside of the typical operating range for that parameter.

In some embodiments, the graphical interface, and any associated inputs such as buttons, may be used to operate the needle-free injector and/or to provide operational information during use. For example, for a needle-free injector having buttons near the graphical interface, the buttons may be used to select one or more modes of operation, such as powering the needle-free injector on from a powered down state and/or turn on or off the at least one light source. In further embodiments, the graphical interface may include an indicator light configured to provide, at a glance, status information during use of the needle-free injector. For example, the indicator light may illuminate when the battery is lower than a threshold charge.

The graphical interface may be configured to display analytical or cumulative injection information, including but not limited to, drug contents and amounts, a number of subjects who have received injections, information extracted from a detectable marker, a volume of fluid remaining in the at least one source of injectate and a number of subjects remaining to receive a volume of fluid. This information may aid an end user or operator in performing injections, improve process efficiency, and reduce waste/costs associated with excess injectate usage.

### Data Communication

A system featuring a needle-free injector as described herein may include a local buffer of injections so that a user does not need a network to make injection decisions, such as whether a human or animal has been injected previously. In this context, human or animal injection histories may be loaded onto the injector before a dosing regimen is initiated, so that the injector can autonomously execute dosing decisions on an case-by-case basis.

In accordance with one or more embodiments, there is provided a needle-free injector. The needle-free injector may include a housing, at least one chamber within the housing constructed and arranged to be fluidly coupled to at least one source of an injectate, and at least one nozzle fluidly coupled to the chamber. The needle-free injector may further include a plunger slidably coupled to and disposed within the chamber, the plunger positioned to discharge a bolus of the at least one injectate through an exit port when slid within the chamber and a motor operatively coupled to the plunger. The motor may be operable to actuate the plunger in the chamber. The needle-free injector may additionally include a controller operatively coupled to the motor. The controller may be operable to operate the plunger according to a first delivery profile configured to inject a first bolus of a first injectate to a first predetermined depth in a subject. The controller may be further operable to operate the plunger according to a second delivery profile configured to inject a second bolus of a second injectate to a second predetermined depth in the subject. The first predetermined depth and the second predetermined depth may be such that the first bolus and the second bolus do not mix at the injection site upon injection into the subject.

In accordance with one or more embodiments, there is provided a needle-free injector. The needle-free injector may include a housing, a plurality of chambers within the housing constructed and arranged to be fluidly coupled to a respective plurality of sources of an injectate, and a plurality of nozzles fluidly coupled to the plurality of chambers. Each of the plurality of nozzles may be associated with a respective one of the plurality of chambers and spaced to prevent mixing of injectates. Each of the plurality of nozzles may include an adjustable exit diameter and an orientation that is adjustable relative to an axis of flow through the plurality of chambers. The needle-free injector may further include a plurality of plungers disposed within a respective plurality of chambers and a motor operatively coupled to the plurality of plungers. Each of the plurality of plungers may be adapted to slide within a respective chamber and discharge a bolus of one of the plurality of injectates from each of the plurality of chambers. The motor may be operable to actuate the plurality of plungers in the plurality of chambers. The needle-free injector may additionally include a controller operatively coupled to the motor. The controller may be operable to selectively operate the plurality of plungers according to a plurality of delivery profiles. Each of the plurality of delivery profiles may be associated with a respective one of the plurality of chambers and configured to adjust at least one of the nozzle exit diameter and nozzle orientation responsive to at least an identity of a subject, a delivery location on the subject, a velocity of the injectate associated with each of the plurality of chambers, and a composition of each of the plurality of injectates.

In accordance with one or more embodiments, there is provided a needle-free injector. The needle-free injector may include a housing, a chamber within the housing constructed and arranged to be fluidly coupled to at least one source of an injectate, and at least one nozzle fluidly coupled to the chamber. The needle-free injector may further include a plunger slidably coupled to and disposed within the chamber and a motor operatively coupled to the plunger, the motor may be operable to actuate the plunger in the chamber, with the plunger positioned to discharge a bolus of injectate from the at least one source of the injectate through an exit port when slid within the chamber. The needle-free injector may further include a sensor configured to acquire a signal from a detectable marker and a marking system configured to apply an identifier on a subject. The needle-free injector may additionally include a controller operatively coupled to the motor. The controller may be operable to selectively operate the plunger according to a delivery profile configured to deliver the bolus of the injectate. Operation of the delivery profile may be responsive at least to receiving a signal from the detectable marker, with the controller being further operable to cause the marking system to apply the identifier to the subject responsive to injecting the bolus of the injectate to the subject.

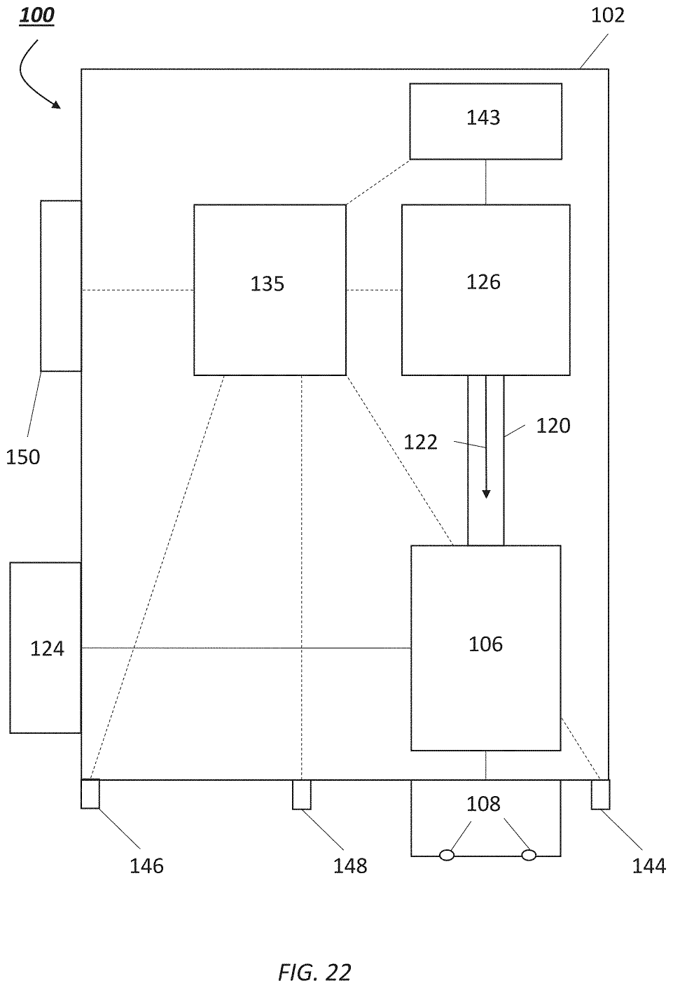

An embodiment of a needle-free injector is illustrated in FIG. 22. With reference to FIG. 22, and in part using the numbering convention of FIG. 1, the needle-free injector 100 includes housing 102 with a chamber 106 having at least one exit port or nozzle 108. While only a single chamber 106 is shown in FIG. 22, the needle-free injector 100 may include a plurality of chambers 106 fluidly coupled to the at least one nozzle 108. In FIG. 22, two nozzles 108 are shown, although it is contemplated that a needle-free injector may include one nozzle or can include more than two nozzles. A plunger 120 is slidably coupled to the chamber 106 and is configured to direct a fluid, such as an injectate, out of the chamber 106 through at least one nozzle 108 along the direction of flow, shown as arrow 122. While only a single plunger 120 is shown in FIG. 20, the needle-free injector 100 may include a plurality of plungers 120 slidably coupled to the plurality of chambers 106. The chamber 106 is fluidly coupled to a reservoir 124 that is configured to provide an injectate to the chamber 106. The plunger 120 is operatively coupled to a motor 126 operable to actuate the plunger 120 in the chamber 106. The motor 126 is further coupled to a power supply 143, such as a battery, that provides electrical power to the motor 126. The motor 126 is additionally coupled to a controller 135 that is operable to selectively operate the plunger 120 according to one or more delivery profiles as described herein.

With continued reference to FIG. 22, the needle-free injector 100 further includes at least one light source 144, positioned near the at least one nozzle 108, that is configured to illuminate a spot on a target or subject. The illuminated spot may provide for increased accuracy for delivering an injectate to the appropriate region on a subject. The needle-free injector further includes a sensor 146 configured to acquire a signal from a detectable marker (not shown). The sensor 146 may be used with the controller 135 to initiate an injection after acquiring information from the detectable marker. The needle-free injector additionally includes a marking system 148. The marking system 148 may be used with the controller 135 to deposit an identifier, such as a spot of paint or ink, onto a surface of a subject following an injection.

With continued reference to FIG. 22, the needle-free injector 100 includes a graphical display 150. The graphical display 150 may be integrated into the housing 102. The graphical display 150 may be coupled to a processor, such as a processor contained on logic boards, that is configured to display to a user a status of the needle-free injector. The graphical display 150 and processor may be configured to receive status information that monitors the status of the various needle-free injector 100 components. The graphical display 150 may be configured to display a status of the needle-free injector 100 that includes at least one of a remaining charge on power supply 143, such as a battery, and service messages for the needle-free injector 100. The graphical display 150 may be further configured to display at least one of a number of subjects who have received injections, information extracted from a detectable marker, a volume of fluid remaining in the at least one source of injectate, such as reservoir 124, and a number of subjects remaining to receive a volume of fluid. The graphical interface 150 may be additionally configured to display a tutorial or training information regarding using the needle-free injector to perform an injection on a subject. For example, the graphical display 150 may provide text-based instructions on a screen of the graphical display 150 or provide pre-recorded spoken commands to guide an end user or operator through the injection process. Alternatively, or in addition, the graphical display 150 may be configured to provide audible and/or visual indicators of the different stages of an injection process. For example, the graphical display may cause a sound to be emitted or cause a light source to change state, for example, pattern or color, when the needle-free injector 100 is ready to inject, or to provide an indication that the subject has already been injected.

In some embodiments, the predetermined depth for each of the boluses of the first injectate and second injectate delivered may be determined by at least one of a velocity of each injectate, an identity of a subject, a species of the subject, a delivery location on the subject, a prescribed regimen for the subject, and a composition of the volume of each injectate. In general, the velocity of the injectate should be such that the stream of the injectate can penetrate an outermost layer of a skin of the subject to access the vascular system beneath the skin. Without being bound by any particular theory, the penetration depth of the injectate may be approximately linearly correlated to the velocity, that is, a greater injectate velocity may lead to a greater penetration depth. The predetermined depth for the injectate may be dependent on the species of the subject. For example, different species may have different skin and tissue thicknesses, and the predetermined depth for one species may not be appropriate of a different species. The predetermined depth may further be dependent on the location that the injectate is to be delivered to on the subject. In general, human or animal skin thickness may vary depending on the region of the body, and skin that is thicker may require a greater injectate velocity in order to access the vasculature underneath at the predetermined depth.

In some embodiments, the predetermined depth for an injectate may be determined by a prescribed regimen for the subject. For example, a subject may be scheduled to receive a plurality of injectates in a specific sequence. Each of the plurality of injectates may be a different type of injectate, e.g., a nutritional supplement or antibiotic, and thus may need to be injected into a different layer of underlying tissue in order to have the expected or desired clinical effect(s) on the subject.

In some embodiments, the predetermined depth for an injectate may be dependent on the composition of the volume of each injectate. As used herein, the composition of the injectate may refer to the chemical properties of the injectate that determine how it is injected into the subject. The chemical properties of the injectate that may determine the predetermined depth includes, but is not limited to, the viscosity, the pH, or the solubility in a particular tissue. As a non-limiting example, an injectate that has a route of administration closer to the upper layers of tissue may have a predetermined depth that is less than an injectate whose route of administration necessitates deeper placement into tissues for efficacy.

Needle-free injectors described herein may include a plurality of chambers that are constructed and arranged to be fluidly coupled to a plurality of sources of injectates. For example, as illustrated in FIGS. 11 and 16-19, a needle-free injector may include two chambers that are connected to two fluid reservoirs that are fixedly secured to an appropriate connector on the housing, that is, bottle-type reservoirs. For needle-free injectors including a plurality of chambers, each of the plurality of chambers may be associated with a plurality of plungers disposed therein. In this configuration, the controller of the needle-free injection may be operable to operate the plurality of plungers according to a plurality of delivery profiles as described herein. For example, each of the plurality of plungers may be discharging a different injectate, whether to a different predetermined depth or of a different composition. This disclosure is not limited by the mechanism of action of each of the plurality of plungers associated with each of the plurality of chambers.

In some embodiments, the at least one nozzle of the needle-free injector may have an adjustable exit diameter, that is, an adjustable exit aperture. The exit diameter of the nozzle may be adjusted by an end user prior to initiating an injection. For example, an end user or the controller of the needle-free injector may set the nozzle exit diameter responsive to at least one of desired injectate velocity, an identity of a subject, a species of the subject, a delivery location on the subject, a prescribed regimen for the subject, a subset of a plurality of the subjects, and a composition of each of the plurality of injectates. Alternatively, the exit diameter of the nozzle may be adjusted automatically by controller. The controller may include information pertaining to the desired velocity, physical and chemical properties, and the subject and, once an end user or operator selects the appropriate information for a specific injection process, the controller may automatically adjust the exit diameter of the nozzle to provide for the correct injectate velocity for that injectate. As a non-limiting example, an injectate of a higher viscosity may require a larger exit diameter of the nozzle in order to achieve the desired injectate velocity without excessive back pressure on the plunger and motor of the needle-free injector. For needle-free injectors having a plurality of chambers associated with a plurality of delivery profiles, each of the plurality of delivery profiles may be configured to adjust the nozzle exit diameter associated with each of the plurality of chambers responsive to at least one of desired injectate velocity, an identity of a subject, a species of the subject, a delivery location on the subject, a prescribed regimen for the subject, a subset of a plurality of the subjects, and a composition of each of the plurality of injectates as described herein.

The adjustable exit dimeter for a nozzle may be from about 50 μm to about 500 μm. For example, the exit diameter for a nozzle may be from about 50 μm to about 500 μm, about 60 μm to about 450 μm, about 70 μm to about 400 μm, about 80 μm to about 350 μm, about 90 μm to about 300 μm, about 100 μm to about 250 μm, or about 200 μm, e.g., about 50 μm, about 60 μm, about 70 μm, about 80 μm, about 90 μm, about 100 μm, about 125 μm, about 150 μm, about 175 μm, about 200 μm, about 225 μm, about 250 μm, about 275 μm, about 300 μm, about 325 μm, about 350 μm, about 375 μm, about 400 μm, about 425 μm, about 450 μm, about 475 μm, or about 500 μm.

In some implementations, the exit diameter of at least one of the plurality of nozzles is different than the remainder of the plurality of nozzles. For example, for a needle-free injector having three nozzles, one of the three nozzles may have a smaller diameter than the other two nozzles. Alternatively, each nozzle may be a different diameter. In further embodiments, each of the plurality of nozzles have the same exit diameter. The disclosure is not limited by the number or the diameter of each of the plurality of nozzles of the needle-free injector.

In some embodiments, the at least one nozzle may have an orientation that is adjustable relative to an axis of flow through the chamber or the plurality of chambers of the device. The adjustment of the nozzle orientation may be responsive to at least velocity of the injectate, an identity of a subject, a delivery location on the subject, and a composition of the volume of the bolus of the injectate. For example, the orientation of the at least one nozzle may be adjusted such that an injectate stream can be directed into a subject at an angle that minimizes the occurrence of the injectate mixing with another injectate; that is, the adjustable nozzles can assist with maintaining the spatial position of the injectate stream. Adjustable nozzles may further aid with injections by allowing an end user or operator to deliver an injectate to a subject with the needle-free injector being in a position that may not be perpendicular to the tissue of the subject. For example, needle-free injector may be held at an angle relative to the tissue surface of the subject; in this configuration, the angle of the nozzle may be adjusted such that the nozzle itself is in the correct orientation to deliver the injectate to the subject.

The adjustable nozzles may have a degree of adjustment that may be about 20 degrees in any direction from the axis of flow through the chamber or the plurality of chambers of the device. For example, the nozzles may have about 1 degree, about 2 degrees, about 3 degrees, about 4 degrees, about 5 degrees, about 6 degrees, about 7 degrees, about 8 degrees, about 9 degrees, about 10 degrees, about 11 degrees, about 12 degrees, about 13 degrees, about 14 degrees, about 15 degrees, about 16 degrees, about 17 degrees, about 18 degrees, about 19 degrees, or about 20 degrees in any direction from the axis of flow through the chamber or the plurality of chambers of the device.

In some embodiments, the needle-free injector may include a sensor that is configured to acquire a signal from a detectable marker. As described herein, a detectable marker may include electromagnetic emitters, e.g., RFID (or similar) data tags, near field communication transceivers, a fiducial marker, e.g., a Quick Response (QR) code, printed to a polymer tag, or the like. The detectable marker may be affixed to an anatomical region of the subject, such as an ear or another feature of their anatomy. As further described herein, detectable markers may be used to uniquely identify a subject for purposes of tracking and retrieval of corresponding information. In some implementations, the detectable markers may directly encode information regarding the subject, e.g., data related to an injection status of the subject, the species of the subject, an injection history of the subject, and a prescribed regimen for the subject. The needle-free injector may include one or more communication features that read from or write to such detectable markers. For example, the needle-free injector may include a sensor, such as an optical sensor, a tag reader or the like, that is configured to acquire a signal from a detectable marker coupled or connected to the subject.

For a needle-free injector equipped with a sensor configured to acquire a signal from a detectable marker, the controller of the needle-free marker may be operable to operate the plunger according to the first delivery profile responsive at least to receiving a signal from the detectable marker. In this configuration, the signal form the detectable marker is acquired to ensure that the correct subject is identified and is being injected with the correct injectate.

In some embodiments, a needle-free injector may include a marking system configured to apply an identifier on the subject. As described herein, the marking system may be used to apply an identifier to a subject following an injection. Thus, in certain embodiments, the needle-free injector may also include a paint or dye source, along with a delivery system that applies a mark to a target animal after an injection is complete. In this configuration, the controller of the needle-free injector may cause the marking system to apply the identifier to the subject responsive only to delivering the bolus of the injectate to the subject. The identifier may provide a rapid visual indicator that a subject has been injected, minimizing potentially dangerous and/or wasteful repeat injections and improving process efficiency.

The controller of a needle-free injector as described herein may be further operable to collect and store data pertaining to at least information extracted from the detectable marker, number of subjects who have received injections, date and time of delivered injections, composition of the injectate, and an aggregate volume of injectate injected. The collected information may be stored on an internal non-volatile memory of the needle-free injection, such as a removable memory device, e.g., a Universal Serial Bus (USB) flash drive or a Secure Digital (SD) card, or a permanently installed memory, for example, a hard drive or solid state memory. The collected and stored data can be transferred from the memory of the needle-free injector using methods known in the art, for example, downloading to a computer or other storage device by way of a connected cable or the like. Alternatively, or in addition, collected and stored data may be transmitted from the device using an appropriate data transmission standard, for example a cellular network, BLUETOOTH® wireless data transmission protocol, or other wireless data transmission protocol known in the art, to a receiver, such as a cellular phone, tablet, computer, server, or the like.

In accordance with another aspect, there is provided a method of delivering a fluid using a needle-free injector. The method may include providing a needle-free injector as described herein and, responsive to initiating an injection with the needle-free injector, causing the needle-free injector to inject a bolus of an injectate into a subject.

In some embodiments, the method may include measuring an aggregate volume of injectate delivered. In some embodiments, the method may include measuring an aggregate count of the number of injectates delivered to a plurality of subjects. After measuring an aggregate count of the number of injectates delivered to a plurality of subjects, the method may include subtracting the aggregate count from an expected total count to determine a remaining population of subjects requiring injection. In further embodiments, responsive to injecting the subject, the method may include applying an identifier to the subject.

In accordance with another aspect, there is provided a method of facilitating needle-free injection of a fluid. The method may include providing a needle-free injector as described herein. In some embodiments, the method may include providing instructions to a user for connecting at least one source of the fluid to the needle-free injector. In some embodiments, the method may include providing instructions to a user for operating the needle-free injector.

The controller of the needle-free injector described herein may be operable to initiate a cleaning cycle responsive to a final delivery of injectate. The cleaning cycle may include discharging a volume of a fluid through the at least one nozzle. The fluid that is used to clean the needle-free injector may be any suitable cleaning fluid, such as a detergent solution or water. The controller may initiate various types of cleaning cycles. For example, the controller may initiate a quick clean or a rinse between one or more injectates during the course of normal operation of the needle-free injector. Alternatively, or in addition, the controller may initiate a more complete cleaning of the needle-free injector after the completion of a preset injection schedule, such as after injecting all of the subjects in a given area or during a given time period. The controller may initiate a more complete cleaning of the needle-free injector under conditions where different types of injectates were delivered to reduce contamination between the different injectates.

It is to be understood that the foregoing description is intended to illustrate and not to limit the scope of the invention, which is defined by the scope of the appended claims. Other embodiments are within the scope of the following claims.

1. A needle-free injector, comprising:
a housing;
at least one chamber within the housing constructed and arranged to be fluidly coupled to at least one source of an injectate;
at least one nozzle fluidly coupled to the chamber;
a plunger slidably coupled to and disposed within the chamber, the plunger positioned to discharge a bolus of the at least one injectate through an exit port when slid within the chamber;
a motor operatively coupled to the plunger, the motor operable to actuate the plunger in the chamber, and
a controller operatively coupled to the motor, the controller operable to operate the plunger according to a first delivery profile, the first delivery profile configured to inject a first bolus of a first injectate to a first predetermined depth in a subject, the controller further operable to operate the plunger according to a second delivery profile configured to inject a second bolus of a second injectate to a second predetermined depth in the subject, wherein the first predetermined depth and the second predetermined depth are such that the first bolus and the second bolus do not mix at the injection site upon injection into the subject.

2. The needle-free injector of claim 1, wherein the predetermined depth for each of the boluses of the first injectate and second injectate delivered is determined by at least one of a velocity of each injectate, an identity of a subject, a species of the subject, a delivery location on the subject, a prescribed regimen for the subject, and a composition of the volume of each injectate.

3. The needle-free injector of claim 2, further comprising a plurality of chambers constructed and arranged to be fluidly coupled to a respective plurality of sources of injectates.

4. The needle-free injector of claim 3, further comprising a plurality of plungers disposed within the respective plurality of chambers.

5. The needle-free injector of claim 1, wherein the at least one nozzle comprises at least one of an adjustable exit diameter and an orientation that is adjustable relative to an axis of flow through the plurality of chambers.

6. The needle-free injector of claim, 1, wherein the controller is further operable to operate the plurality of plungers according to a plurality of delivery profiles.

7. The needle-free injector of claim 6, wherein each of the plurality of delivery profiles is configured to adjust at least one of the nozzle exit diameter and nozzle orientation responsive to at least one of desired injectate velocity, an identity of a subject, a species of the subject, a delivery location on the subject, a prescribed regimen for the subject, a subset of a plurality of the subjects, and a composition of each of the plurality of injectates.

8. The needle-free injector of claim 1, further comprising a marking system configured to apply an identifier on the subject.

9. The needle-free injector of claim 8, further comprising a sensor configured to acquire a signal from a detectable marker.

10. The needle-free injector of claim 9, wherein the controller is further operable to operate the plunger according to the first delivery profile responsive at least to receiving a signal from the detectable marker.

11. The needle-free injector of claim 10, wherein the controller is further operable to cause the marking system to apply the identifier to the subject responsive to delivering the bolus of the injectate to the subject.

12-20. (canceled)

21. A needle-free injector, comprising:
a housing;
a chamber within the housing constructed and arranged to be fluidly coupled to at least one source of an injectate;
at least one nozzle fluidly coupled to the chamber;
a plunger slidably coupled to and disposed within the chamber, the plunger positioned to discharge a bolus of injectate from the at least one source of the injectate through an exit port when slid within the chamber;
a motor operatively coupled to the plunger, the motor operable to actuate the plunger in the chamber,
a sensor configured to acquire a signal from a detectable marker;
a marking system configured to apply an identifier on a subject; and
a controller operatively coupled to the motor, the controller operable to selectively operate the plunger according to a delivery profile configured to deliver the bolus of the injectate, operation of the delivery profile responsive at least to receiving a signal from the detectable marker, the controller further operable to cause the marking system to apply the identifier to the subject responsive to injecting the bolus of the injectate to the subject.

22. The needle-free injector of claim 21, wherein the chamber is configured to be fluidly coupled to a plurality of sources of injectate.

23. The needle-free injector of claim 21, further comprising a plurality of chambers.

24. The needle-free injector of claim 21, wherein the at least one nozzle comprises at least one of an adjustable exit diameter and an orientation that is adjustable relative to an axis of flow through the chamber.

25. The needle-free injector of claim 24, wherein the controller is further operable to adjust at least one of the nozzle exit diameter and nozzle orientation responsive to at least one of an identity of a subject, a species of the subject, a delivery location on the subject, a prescribed regimen for the subject, a subset of a plurality of the subjects, and a composition of the bolus of the injectate.

26. The needle-free injector of claim 25, wherein the adjustment to at least one of the nozzle exit diameter and nozzle orientation is responsive to at least velocity of the injectate, an identity of a subject, a delivery location on the subject, and a composition of the volume of the bolus of the injectate.

27. The needle-free injector of claim 23, wherein the controller is further operable to operate the plunger to deliver a plurality of boluses of injectates from the respective plurality of chambers to a predetermined depth, the predetermined depth for each of the injectates are such that each bolus delivered does not mix at the injection site upon injection into the subject.

28. The needle-free injector of claim 27, wherein the predetermined depth for each of the boluses of the injectates is determined by at least a velocity of the injectate, an identity of a subject, a delivery location on the subject, and a composition of the bolus of the injectate.

29. The needle-free injector of claim 21, wherein the detectable marker comprises an electromagnetic emitter.

30. The needle-free injector of claim 21, wherein the detectable marker comprises a fiducial marker.

31. The needle-free injector of claim 1, wherein the source of the injectate comprises a reservoir fixedly connected to the housing.

32. The needle-free injector of claim 1, wherein a source of the injectate comprises a reservoir fluidly coupled to the housing by a flexible conduit.

33. The needle-free injector of claim 1, further comprising at least one light source constructed and arranged to illuminate a mark onto an area of the subject.

34. The needle-free injector of claim 1, further comprising a graphical display integrated into the housing operatively coupled to the controller, the graphical display configured to display to a user information pertaining to at least one of the status of the needle-free injector and an injection process.

35. The needle-free injector of claim 34, wherein the graphical display is configured to display a status of the needle-free injector that includes at least one of a remaining charge on a battery and service messages for the needle-free injector.

36. The needle-free injector of claim 35, wherein the graphical display is configured to display at least one of a number of subjects who have received injections, information extracted from a detectable marker, a volume of fluid remaining in the at least one source of injectate and a number of subjects remaining to receive a volume of fluid.

37. The needle-free injector of claim 1, wherein the controller is further operable to initiate a cleaning cycle responsive to a final delivery of injectate, the cleaning cycle comprising discharging a volume of a fluid through the at least one nozzle.

38. The needle-free injector of claim 1, wherein the controller is further operable to collect and store data pertaining to at least information extracted from the detectable marker, number of subjects who have received injections, date and time of delivered injections, composition of the injectate, and an aggregate volume of injectate injected.

39. A method of delivering a fluid using a needle-free injector, the method comprising:
providing the needle-free injector of claim 1; and
responsive to initiating an injection with the needle-free injector, causing the needle-free injector to inject a bolus of an injectate into a subject.

40. The method of claim 39, further comprising, prior to initiating an injection, identifying a subject in need thereof by extracting information from a detectable marker coupled to the subject.

41. The method of claim 39, further comprising measuring an aggregate volume of injectate delivered.

42. The method of claim 39, further comprising measuring an aggregate count of the number of injectates delivered to a plurality of subjects.

43. The method of claim 42, further comprising subtracting the aggregate count from an expected total count to determine a remaining population of subjects requiring injection.

44. The method of claim 39, further comprising applying an identifier to the subject responsive to injecting the subject.

45. A method of facilitating needle-free injection of a fluid, the method comprising providing the needle-free injector of claim 1.

46. The method of claim 45, further comprising providing instructions to a user for connecting at least one source of the fluid to the needle-free injector.

47. The method of claim 45, further comprising providing instructions to a user for operating the needle-free injector.
